(19) United States
(12) Patent Application Publication
(10) Pub. No.: US 2025/0243466 A1
(43) Pub. Date: Jul. 31, 2025

(54) METHODS AND COMPOSITIONS FOR CELL CULTURING

(71) Applicant: Uncommon Bio Limited, Cambridge (GB)

(72) Inventors: Ruth Helen Faram, Ely (GB); Ariane Standing, Carshalton Surrey (GB)

(21) Appl. No.: 19/035,539

(22) Filed: Jan. 23, 2025

Related U.S. Application Data
(63) Continuation of application No. PCT/GB2023/051994, filed on Jul. 27, 2023.

(30) Foreign Application Priority Data
Jul. 28, 2022 (GB) 2211072.0

(51) Int. Cl. C12N 5/074

(52) U.S. Cl. CPC C12N 5/0696; C12N 2501/115; C12N 2510/00

(57) ABSTRACT

Provided herein are methods and compositions for growing cells. The cells may comprise engineered nucleic acids. The methods provided herein may comprise growing the cells comprising the engineered nucleic acids, or a progeny or population of cells derived thereof, within a culture medium. The culture medium provided herein may comprise a limited amount or without a growth factor. Also provided herein, are compositions or kits to practice the methods. The compositions generated or derived from the cells or growth of the same are also provided.

### CROSS-REFERENCE

This application is a continuation of International Patent Application No. PCT/GB2023/051994, filed Jul. 27, 2023, which claims the benefit of United Kingdom Patent Application No. 2211072.0, filed Jul. 28, 2022, each of which is entirely incorporated herein by reference.

### BACKGROUND

Eukaryotic cells, whether engineered or not, can synthesize a variety of molecules or be converted into products for various uses, including therapeutic production, food production, research, or industrial application. These molecules or products can comprise proteins, peptides, or metabolites synthesized by the cell. In some cases, the cell can also be used in these various purposes. These molecules can be harvested after the growth of a relatively large number of cells and the synthesis of molecules by the cells. In some cases, the cells can be used directly for the aforementioned uses without having to harvest the molecules produced by the cells, including but not limited to cultivated meat and cell therapy. Efficient and cost-effective culturing of the cells can facilitate such uses. However, currently available culturing methods can be limited due to a lack of effectiveness, scalability, or reliability. Obtaining the growth-factors by chemical or in vitro synthesis can be costly and technically challenging. Additionally, using animal serum to supplement the growth-factors or other molecules to support growth and culturing of cells in vitro or ex vivo may require slaughtering a large number of animals. Furthermore, the current available culturing methods may require genetic modification of the cells in which the genetic modification could pass down to the progenies or derivatives of the cells.

### SUMMARY

Provided herein are methods and compositions for culturing cells. The cells may comprise or be induced to comprise at least an engineered nucleic acid. The engineered nucleic acid may encode a polypeptide. The polypeptide may comprise a cell proliferation regulator. The cell proliferation regulator may allow the cell to proliferate within a culture medium comprising an amount of growth-factors insufficient for growing the cell without the engineered nucleic acid. The engineered nucleic acid, the peptide or polypeptide encoded by the engineered nucleic acid, or a molecule generated by the cell comprising the engineered nucleic acid may substitute for the growth-factors in the culture medium. In some cases, a conditioned medi may be generated by a cell comprising the engineered nucleic acid, and wherein the conditioned be used for growing another cell. The engineered nucleic acid, the peptide or polypeptide encoded by the engineered nucleic acid, or a molecule generated by the cell comprising the engineered nucleic acid may also bypass the growth-factor requirement for the cells. Hence, the engineered nucleic acid, the peptide or polypeptide encoded by the engineered nucleic acid, or a molecule generated by the cell comprising the engineered nucleic acid may reduce the amount of or eliminate the growth-factors in the culture medium for culturing the cells. The methods and compositions provided herein may provide various approaches for effective, scalable, or reliable culturing of eukaryotic cells, thereby facilitating the production of material (e.g., cells or molecules) for various applications, such as therapeutics applications, food applications, research applications or industrial applications.

In an aspect, provided here is a method comprising: (a) providing a cell comprising an engineered ribonucleic acid (RNA); and (b) growing the cell within a culture medium, wherein the culture medium comprises at most about 100 micrograms per milliliter (μg/mL) exogenous growth-factor.

In some embodiments, the engineered RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), a piwi-interacting RNA (piRNA), a short hairpin RNA (shRNA), a transacting RNA (tasiRNA), a long non-coding RNA (lncRNA), a cis-natural antisense transcript (cis-NAT), or a combination thereof. In some embodiments, the engineered RNA comprises the saRNA. In some embodiments, the method further comprises expressing a polypeptide encoded by the engineered RNA, or a derivative thereof, by the cell. In some embodiments, the method further comprises harvesting the polypeptide expressed by the engineered RNA or the derivative thereof from the culture medium. In some embodiments, the method further comprises contacting the cell with the engineered RNA. In some embodiments, the contacting comprises transfecting the cell with the engineered RNA. In some embodiments, the growing in (b) generates a population of the cell. In some embodiments, the population of the cell comprises at least a progeny of the cell. In some embodiments, the method further comprises growing the at least the progeny of the cell within the culture medium. In some embodiments, the progeny of the cell proliferates within the culture medium by using a polypeptide derived from the cell. In some embodiments, the cell secretes the polypeptide derived from the cell. In some embodiments, the polypeptide derived from the cell is encapsulated by an endogenous micro-vesicle derived from the cell. In some embodiments, the endogenous micro-vesicle is an exosome. In some embodiments, the engineered RNA encodes one or more polypeptides comprising at least a sequence comprising at least 70% sequence identity to the exogenous growth-factor. In some embodiments, the engineered RNA encodes one or more polypeptides comprising at least a sequence comprising at least 70% sequence identity to a growth-factor receptor activated by the exogenous growth-factor. In some embodiments, the one or more polypeptides are an activated form of the growth-factor receptor. In some embodiments, the activated form of the growth-factor receptor is configured to be activated in an absence of a growth-factor. In some embodiments, the growth-factor comprises the exogenous growth-factor. In some embodiments, the engineered RNA encodes a polypeptide comprising a growth-factor, a growth-factor receptor, a cell proliferation signaling pathway regulator, a cell-cycle regulator, an immune response regulator, a Maillard reaction peptide, a metallopeptide, or a combination thereof. In some embodiments, the Maillard reaction peptide comprises a lysine-rich polypeptide, a cysteine-rich polypeptide, or an arginine-rich polypeptide, or a combination thereof. In some embodiments, the engineered RNA encodes Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof. In some embodiments, the cell comprises at least two engineered RNAs. In some embodiments, the at least two engineered RNAs are different. In some embodiments, the at least two engineered RNAs are copies of a same engineered RNA. In some embodiments, the engineered RNA comprises an alphavirus replication sequence, a Kozac sequence, a poly-A tail, a 5′cap, an internal Ribosome Entry Sequence (IRES), a sequence encoding a 2A self-cleaving peptide, or a 26S subgenomic promoter, or a combination thereof. In some embodiments, the engineered RNA comprises at most one copy of a coding sequence. In some embodiments, the engineered RNA comprises at least two copies of a coding sequence. In some embodiments, the engineered RNA further comprises a sequence encoding a cleavage site between the at least two copies of the coding sequence.

In an aspect, provided herein is a composition comprising: (a) a culture medium; and (b) an engineered ribonucleic acid (RNA), wherein the culture medium comprises at most about 100 micrograms per milliliter (μg/mL) exogenous growth-factor.

In some embodiments, the engineered RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), a piwi-interacting RNA (piRNA), a short hairpin RNA (shRNA), a transacting RNA (tasiRNA), a long non-coding RNA (lncRNA), a cis-natural antisense transcript (cis-NAT), or a combination thereof. In some embodiments, the engineered RNA comprises the saRNA. In some embodiments, the engineered RNA encodes one or more polypeptides comprising at least a sequence comprising at least 70% sequence identity to the exogenous growth-factor. In some embodiments, the engineered RNA encodes one or more polypeptides comprising at least a sequence comprising at least 70% sequence identity to a growth-factor receptor activated by the exogenous growth-factor. In some embodiments, the one or more polypeptides are an activated form of the growth-factor receptor. In some embodiments, the activated form of the growth-factor receptor is configured to be activated in an absence of a growth-factor. In some embodiments, the growth-factor comprises the exogenous growth-factor. In some embodiments, the engineered RNA encodes a polypeptide comprising a growth-factor, a growth-factor receptor, a cell proliferation signaling pathway regulator, a cell-cycle regulator, an immune response regulator, a Maillard reaction peptide, a metallopeptide, or a combination thereof. In some embodiments, the Maillard-reaction peptide comprises a lysine-rich polypeptide, a cysteine-rich polypeptide, or an arginine-rich polypeptide, or a combination thereof. In some embodiments, the engineered RNA encodes Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof. In some embodiments, the cell comprises at least two engineered RNAs. In some embodiments, the at least two engineered RNAs are different. In some embodiments, the at least two engineered RNAs are copies of a same engineered RNA. In some embodiments, the engineered RNA comprises an alphavirus replication sequence, a Kozac sequence, a poly-A tail, a 5′cap, an internal Ribosome Entry Sequence (IRES), a sequence encoding a 2A self-cleaving peptide, or a 26S subgenomic promoter, or a combination thereof. In some embodiments, the engineered RNA comprises at most one copy of a coding sequence. In some embodiments, the engineered RNA comprises at least two copies of a coding sequence. In some embodiments, the engineered RNA further comprises a sequence encoding a cleavage site between the at least two copies of the coding sequence.

In an aspect, provided herein is a method comprising: (a) contacting a cell with an engineered nucleic acid; and (b) growing the cell with a culture medium, wherein the culture medium comprises at most about 100 micrograms per milliliter (μg/mL) exogenous growth-factor, and wherein the engineered nucleic acid encodes a polypeptide that allows the cell to proliferate within the culture medium.

In some embodiments, the method further comprises transfecting the cell with the engineered nucleic acid. In some embodiments, the engineered nucleic acid or a derivative thereof is not integrated into a genome of the cell. In some embodiments, the method further comprises expressing the polypeptide by the cell. In some embodiments, the method further comprises secreting the polypeptide into the culture medium by the cell. In some embodiments, the method further comprises harvesting the polypeptide from the culture medium. In some embodiments, (b) comprises generating a population of the cell. In some embodiments, the population of the cell comprises at least a progeny of the cell. In some embodiments, the method further comprises growing the progeny of the cell within the culture medium. In some embodiments, the progeny of the cell proliferates within the culture medium by using a second polypeptide derived from the cell. In some embodiments, the cell secretes the second polypeptide. In some embodiments, the second polypeptide secreted by the cell is encapsulated by an endogenous micro-vesicle derived from the cell. In some embodiments, the endogenous micro-vesicle is an endogenous exosome. In some embodiments, the polypeptide and the second polypeptide comprise a same polypeptide sequence. In some embodiments, the polypeptide and the second polypeptide comprise different polypeptide sequences. In some embodiments, the engineered nucleic acid is not encapsulated by a micro-vesicle. In some embodiments, the micro-vesicle is an extracellular vesicle. In some embodiments, the polypeptide comprises a growth-factor, a growth-factor receptor, a cell proliferation signaling pathway regulator, a Maillard reaction peptide, a metallopeptide, a cell-cycle regulator, an immune response regulator, or a combination thereof. In some embodiments, the polypeptide comprises the growth-factor. In some embodiments, the growth-factor and the exogenous growth-factor are a same growth-factor. In some embodiments, the growth-factor and the exogenous growth-factor are different. In some embodiments, the polypeptide comprises the growth-factor receptor. In some embodiments, the growth-factor receptor is an activated form of an endogenous growth-factor of the cell. In some embodiments, the endogenous growth-factor of the cell is activated by the exogenous growth-factor. In some embodiments, the growth-factor receptor is activated in an absence of the exogenous growth-factor. In some embodiments, the endogenous growth-factor of the cell is activated by a second growth-factor different from the exogenous growth-factor. In some embodiments, the growth-factor receptor is activated in an absence of the second growth-factor. In some embodiments, the Maillard-reaction peptide comprises a lysine-rich polypeptide, a cysteine-rich polypeptide, or an arginine-rich polypeptide, or a combination thereof. In some embodiments, the polypeptide comprises Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof.

In an aspect, provided herein is a composition comprising: (a) an engineered nucleic acid; (b) a cell; and (c) a culture medium, wherein the culture medium comprises at most about 100 micrograms per milliliter (μg/mL) exogenous growth-factor, and wherein the engineered nucleic acid encodes a polypeptide that is configured to allow the cell to proliferate within the culture medium.

In some embodiments, the engineered nucleic acid or a derivative thereof is configured not to be integrated into a genome of the cell. In some embodiments, the engineered nucleic acid is not encapsulated by a micro-vesicle. In some embodiments, the engineered nucleic acid comprising an engineered RNA comprising a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), a piwi-interacting RNA (piRNA), a short hairpin RNA (shRNA), a transacting RNA (tasiRNA), a long non-coding RNA (lncRNA), a cis-natural antisense transcript (cis-NAT), or a combination thereof. In some embodiments, the polypeptide comprises a growth-factor, a growth-factor receptor, a cell proliferation signaling pathway regulator, a Maillard reaction peptide, a metallopeptide, a cell-cycle regulator, an immune response regulator, or a combination thereof. In some embodiments, the polypeptide comprises the growth-factor. In some embodiments, the growth-factor and the exogenous growth-factor are a same growth-factor. In some embodiments, the growth-factor and the exogenous growth-factor are different. In some embodiments, the polypeptide comprises the growth-factor receptor. In some embodiments, the polypeptide is an activated form of an endogenous growth-factor of the cell. In some embodiments, the endogenous growth-factor of the cell is activated by the exogenous growth-factor. In some embodiments, the growth-factor receptor is activated in an absence of the exogenous growth-factor. In some embodiments, the endogenous growth-factor of the cell is activated by a second growth-factor different from the exogenous growth-factor. In some embodiments, the growth-factor receptor is activated in an absence of the second growth-factor. In some embodiments, the Maillard-reaction peptide comprises a lysine-rich polypeptide, a cysteine-rich polypeptide, or an arginine-rich polypeptide, or a combination thereof. In some embodiments, the polypeptide comprises Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof.

In an aspect, provided herein is a composition comprising: (a) an engineered ribonucleic acid (RNA), wherein the engineered RNA encodes a polypeptide; and (b) a cell, wherein the engineered RNA is configured to allow the cell to proliferate for at least 1 cell division when present within a culture medium without exogenous growth-factors.

In some embodiments, the engineered RNA is configured to allow the cell to proliferate for at least 5 cell divisions when present within the culture medium without the exogenous growth-factors. In some embodiments, the engineered RNA is configured to allow the cell to proliferate for at least 10 cell divisions when present within the culture medium without the exogenous growth-factors.

In an aspect, provided herein is a composition comprising: (a) an engineered ribonucleic acid (RNA), wherein the engineered RNA encodes a polypeptide; and (b) a cell, wherein the engineered RNA is configured to allow the cell, when present within a culture medium without exogenous growth-factors, to exhibit a proliferation rate at least about 110% relative to a proliferation rate of the cell that does not comprise the engineered RNA when present within the culture medium in an absence of the exogenous growth-factors.

In some embodiments, the engineered RNA is configured to allow the cell, when present within the culture medium without the exogenous growth-factors, to exhibit a proliferation rate at least about 150% relative to the proliferation rate of the cell that does not comprise the engineered RNA when present within the culture medium in the absence of the exogenous growth-factors. In some embodiments, the engineered RNA is configured to allow the cell, when present within the culture medium without the exogenous growth-factors, to exhibit a proliferation rate at least about 500% relative to the proliferation rate of the cell that does not comprise the engineered RNA when present within the culture medium in the absence of the exogenous growth-factors. In some embodiments, the engineered RNA is not encapsulated by a micro-vesicle. In some embodiments, the engineered RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), a piwi-interacting RNA (piRNA), a short hairpin RNA (shRNA), a transacting RNA (tasiRNA), a long non-coding RNA (lncRNA), a cis-natural antisense transcript (cis-NAT), or a combination thereof. In some embodiments, the engineered RNA comprises the saRNA. In some embodiments, the engineered RNA or a derivative thereof is configured not to be integrated into a genome of the cell. In some embodiments, the polypeptide comprises a growth-factor, a growth-factor receptor, a cell proliferation signaling pathway regulator, a cell-cycle regulator, an immune response regulator, a Maillard-reaction peptide, a metallopeptide, or a combination thereof. In some embodiments, the polypeptide comprises the growth-factor. In some embodiments, the growth-factor and an exogenous growth-factor of the exogenous growth-factors are a same growth-factor. In some embodiments, the growth-factor and an exogenous growth-factor of the exogenous growth-factors are different. In some embodiments, the polypeptide comprises the growth-factor receptor. In some embodiments, the polypeptide is an activated form of an endogenous growth-factor receptor of the cell. In some embodiments, the endogenous growth-factor receptor of the cell is configured to be activated by an exogenous growth-factor of the exogenous growth-factors. In some embodiments, the growth-factor receptor is configured to be activated in an absence of the exogenous growth-factors. In some embodiments, the polypeptide is an activated form of a growth-factor receptor not expressed by the cell that does not comprise the engineered RNA. In some embodiments, the growth-factor receptor is configured to be activated by an exogenous growth-factor of the exogenous growth-factors. In some embodiments, the polypeptide is configured to be activated in an absence of the exogenous growth-factors. In some embodiments, the Maillard-reaction peptide comprises a lysine-rich polypeptide, a cysteine-rich polypeptide, or an arginine-rich polypeptide, or a combination thereof. In some embodiments, the polypeptide comprises Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof.

In an aspect, provided herein is a method comprising: (a) providing a cell comprising an engineered ribonucleic acid (RNA), wherein the engineered RNA encodes a growth-factor, wherein the engineered RNA is synthesized in vitro; and (b) growing the cell within a culture medium, wherein the culture medium comprises at most about 100 micrograms per milliliter (μg/mL) of an exogenous polypeptide comprising a sequence comprising at least about 70% sequence identity to the growth-factor.

In some embodiments, the sequence comprises at least about 90% sequence identity to the growth-factor. In some embodiments, the sequence comprises 100% sequence identity to the growth-factor. In some embodiments, the culture medium comprises at most about 50 μg/mL of the exogenous polypeptide. In some embodiments, the engineered RNA is synthesized by an in vitro transcription or an in vitro synthesis. In some embodiments, the engineered RNA is synthesized by the in vitro transcription. In some embodiments, the engineered RNA is transcribed from a DNA template In some embodiments, the engineered RNA is transcribed from a plasmid template. In some embodiments, the engineered RNA is transcribed from an RNA promotor. In some embodiments, the promotor comprises a T7 promotor, a T3 promotor, a SP6 promotor, or a combination thereof. In some embodiments, the engineered RNA or a derivative thereof is not integrated into a genome of the cell.

In an aspect, provided herein is a method comprising: (a) growing a cell within a culture medium; and (b) converting the cell into a meat product, wherein the culture medium comprises at most about 100 micrograms per milliliter (μg/mL) of an exogenous growth-factor.

In some embodiments, the cell comprises an engineered nucleic acid. In some embodiments, the engineered nucleic acid encodes a polypeptide. In some embodiments, the method further comprises, prior to (a), contacting the cell with an engineered nucleic acid. In some embodiments, the engineered nucleic acid encodes a polypeptide. In some embodiments, the contacting comprises transfecting the cell with the engineered nucleic acid. In some embodiments, the meat product comprises a muscle cell, a fat cell, a connective tissue cell, a vasculature cell, a neuron, a bone cell, or a skin cell, or a combination thereof. In some embodiments, the culture medium comprises a serum-free medium. In some embodiments, the culture medium does not comprise the exogenous growth-factor. In some embodiments, the exogenous growth-factor comprises a peptide. In some embodiments, the exogenous growth-factor does not comprise a peptide. In some embodiments, (a) further comprises generating a population of the cell. In some embodiments, the population of the cell comprises at least a progeny of the cell. In some embodiments, the method further comprises growing the at least the progeny of the cell within the culture medium. In some embodiments, the at least the progeny of the cell proliferates within the culture medium by using a second polypeptide derived from the cell. In some embodiments, the cell secretes the second polypeptide. In some embodiments, the second polypeptide is encapsulated by an endogenous micro-vesicle derived from the cell. In some embodiments, the endogenous micro-vesicle is an exosome.

In some embodiments, the cell is a differentiated cell. In some embodiments, the cell is not a stem cell. In some embodiments, the cell comprises a stem cell. In some embodiments, the stem cell comprises an induced pluripotent stem cell (iPSC).

In some embodiments, the cell comprises a muscle cell, a fat cell, a connective tissue cell, a vasculature cell, a neuron, a bone cell, a skin cell, or a combination thereof. In some embodiments, the cell is a differentiated cell or a stem cell. In some embodiments, the cell is the differentiated cell. In some embodiments, the cell is a terminally differentiated cell. In some embodiments, the cell is the stem cell. In some embodiments, the stem cell comprises an induced pluripotent stem cell (iPSC).

In an aspect, provided herein is a method comprising: (a) providing a cell comprising an engineered self-amplifying ribonucleic acid (saRNA); and (b) growing the cell within a culture medium, wherein the culture medium comprises at most about 100 micrograms per milliliter (μg/mL) exogenous growth-factor.

In an aspect, provided herein is a composition comprising: (a) a culture medium; and (b) an engineered self-amplifying ribonucleic acid (saRNA), wherein the culture medium comprises at most about 100 micrograms per milliliter (μg/mL) exogenous growth-factor.

In an aspect, provided herein is a composition comprising: (a) an engineered ribonucleic acid (RNA), wherein the engineered RNA encodes a polypeptide, wherein the engineered RNA is not encapsulated by a micro-vesicle; and (b) a cell, wherein the engineered RNA is configured to allow the cell to proliferate for at least 1 cell division when present within a culture medium without exogenous growth-factors.

In an aspect, provided herein is a method comprising: (a) contacting a cell with an engineered nucleic acid, wherein the engineered nucleic acid is not encapsulated by a micro-vesicle; and (b) growing the cell with a culture medium, wherein the culture medium comprises at most about 100 micrograms per milliliter (μg/mL) exogenous growth-factor, and wherein the engineered nucleic acid encodes a polypeptide that allows the cell to proliferate within the culture medium.

In an aspect, provided herein is a composition comprising: (a) an engineered nucleic acid, wherein the engineered nucleic acid is not encapsulated by a micro-vesicle; (b) a cell; and (c) a culture medium, wherein the culture medium comprises at most about 100 micrograms per milliliter (μg/mL) exogenous growth-factor, and wherein the engineered nucleic acid encodes a polypeptide that is configured to allow the cell to proliferate within the culture medium.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (“FIG.” or “FIGs.” herein), of which:

FIG. 1 depicts an example vector comprising an engineered nucleic acid that comprises one polypeptide sequence.

FIG. 2 depicts an example vector comprising an engineered nucleic acid that comprises multiple polypeptide sequences.

FIG. 3 depicts western blot analysis showing cells transfected with various amounts of engineered nucleic acids expressing the polypeptides encoded by the engineered nucleic acids.

FIG. 4A depicts transmitted light and GFP fluorescence images of cells transfected with various engineered nucleic acids.

FIG. 4B depicts the quantification of the number of the cells of FIG. 4A growing without an exemplary growth factor.

FIG. 4C depicts the quantification of an exemplary polypeptide secreted into the culture media by the cells of FIG. 4A

FIG. 5A depicts transmitted light and GFP fluorescence images of cells cultured within the conditioned medium generated by cells comprising an exemplary engineered nucleic acid.

FIG. 5B depicts the quantification of cell proliferation of the cells of FIG. 5A.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “cell proliferation,” as used herein, generally refers to a process by which a cell increases its numbers, mass, or a combination thereof.

The term “cell proliferation regulator,” as used herein, generally refers to a cellular component that is involved in a proliferation of a cell (or a population thereof) or cell proliferation.

The term “culture medium” or “culture media,” as used herein, generally refers to the combination of solvents, molecules, compounds, metabolites, nutrients, and/or chemicals for supporting the growth of a cell or maintenance of viability of a cell in vitro or ex vivo.

The term “endogenous,” as used herein, generally when referring to a molecule or a plurality of molecules of a cell, generally refers to a molecule or a plurality of molecules that is produced by the cell that has not been engineered.

The term “engineered cell,” as used herein, generally refers to a cell that has undergone modification with viral or non-viral means.

The term “engineered nucleic acid” or “engineered,” as used herein when referring to a type of nucleic acid, generally refers to a non-naturally occurring nucleic acid.

The term “exogenous,” as used herein when referring to a molecule present within a culture medium for culturing a cell, generally refers to a molecule that is not produced by the cell.

The term “meat” or “meat product,” as used herein, generally refers to a group of cells for consumption.

As used herein and thereof, the singular forms “a,” “an,” and “the” include plural references unless the context clearly dictates otherwise.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

### Overview

The present disclosure provides methods, compositions, and/or kits for culturing a cell. The methods, compositions, and/or kits provided herein can allow culturing cells with a limited amount of exogenous growth factor(s) or without the growth factor(s). The methods, compositions, and/or kits provided herein can thus omit obtaining the growth-factors by chemical or in vitro synthesis (or significantly reduce the amounts of the growth-factors) that can be costly and technically challenging. For example, the methods, compositions, and/or kits provided herein can reduce or eliminate the process of purifying the growth factors synthesized by chemical or in vitro synthesis. Additionally, the methods, compositions, and/or kits provided herein can reduce or eliminate the uses of animal serum to supplement the growth-factors or other molecules to support growth and culturing of cells in vitro or ex vivo. The methods, compositions, and/or kits provided herein can also reduce or eliminate slaughtering a large number of animals. Furthermore, the methods, compositions, and/or kits provided herein can bypass using genetic modification of the cells such that the progenies or derivatives of the cells would also not be genetically modified. Thus, the methods, compositions, and/or kits provided herein can provide significantly increased effectiveness, scalability, and/or reliability over the currently available methods, compositions, and/or kits.

The method may comprise providing at least a cell with at least a nucleic acid. A method may comprise growing the cell comprising the nucleic acid within a culture medium. The culture medium may comprise an amount of at least a growth-factor or exogenous growth-factor, wherein the amount of the growth-factor or exogenous growth-factor may be insufficient for the growth of a cell without the nucleic acid (i.e., a limited amount of the growth-factors or exogenous growth-factors). The culture medium may not comprise at least a growth-factor or exogenous growth-factor. A cell may comprise the nucleic acid. In some cases, the method may comprise contacting the cell with the nucleic acid. In some instances, the contacting may comprise transfecting the cell with the nucleic acid. In some instances, the contacting may comprise transducing or transforming the cell with the nucleic acid. In some instances, the nucleic acid contacting the cell may not be encapsulated by a micro-vesicle. In some instances, the method may comprise harvesting a cell or a molecule synthesized by a cell. The method may comprise synthesizing a molecule produced by the cell. The cell comprising the nucleic acid may express at least a polypeptide. The polypeptide may be encoded by the nucleic acid. In some cases, the method may comprise expressing a polypeptide by the cell. The method may comprise harvesting a molecule derived from the cell. In some cases, the method may comprise growing at least two cells, wherein a first cell of the at least two cells may comprise an engineered nucleic acid. The second cell of the at least two cells may be a same cell type as the first cell. The second cell may be different from the first cell (e.g., the second cell may be a different cell type of the first cell, or the second cell may not comprise the engineered nucleic acid). In some cases, the second cell may not comprise the engineered nucleic acid or any other engineered nucleic acids. The second cell may proliferate in the presence of the first cell or a molecule synthesized by the first cell. The molecule synthesized by the first cell may comprise a peptide or polypeptide encoded by the engineered nucleic acid. The molecule may also not comprise the peptide or polypeptide encoded by the engineered nucleic acid. For example, the first cell may generate a molecule via an alteration by the engineered nucleic acid or the peptide or polypeptide encoded by thereof. When proliferating the second cell in the presence of the first cell, the method may not comprise purifying the molecule generated from the first cell. The method may also comprise harvesting the molecule derived from the cell. The method may also comprise harvesting the polypeptide derived from the cell. In some cases, the method may comprise generating or obtaining a conditioned medium that has been used to culture or grow the first cell. The conditioned medium may comprise the molecule or the polypeptide (or peptide) synthesized by the first cell. The molecule or polypeptide (or peptide) may be secreted by the cell. A molecule or polypeptide (or peptide) may not be secreted by the cell. In some cases, the molecule or polypeptide (or peptide) secreted by the cell may be encapsulated by an endogenous vesicle. In some cases, the growing of the method may comprise a proliferation of a cell. The proliferation may generate at least a progeny of the cell. In other instances, the method may comprise harvesting the cell, progenies thereof, derivatives thereof, or a combination thereof. In some cases, the method may comprise converting the cell, progenies thereof, derivatives thereof, or a combination thereof into a meat product. In some cases, the compositions may comprise any materials or any combinations of materials for practicing the methods; or the products, materials, derivatives thereof, or any combinations thereof generated by the methods. In some cases, the kits may comprise any materials or combination of the compositions for practicing the methods.

### Nucleic Acid

In some instances, a nucleic acid may be a nucleic acid molecule. In some cases, a nucleic acid may be a species/type of nucleic acid. In some cases, a nucleic acid may comprise a polymeric form of nucleotides. In some cases, a nucleic acid may comprise a polynucleotide. In some cases, the nucleic acid may comprise a sequence of nucleotides (i.e., a nucleic acid sequence). In some cases, a nucleic acid may comprise a modified polynucleotide. In some cases, a nucleic acid may comprise a canonical or non-canonical nucleotide. A canonical nucleotide may comprise adenosine with base types (A), cytosine (C), guanine (G), thymine (T), uracil (U), or variants thereof. When referring to a base type of a nucleotide or a polynucleotide, T and U may be interchangeable. When referring to a sequence of a nucleic acid, the sequence may comprise the complementary form of the sequence. The complementary of the nucleic acid sequence may be based on canonical base-pairing of the nucleotides or nucleic acids.

In some instances, a nucleic acid may comprise an engineered nucleic acid. An engineered nucleic acid may comprise one or more modified nucleotides or nucleotide analogs.

In some cases, an engineered nucleic acid may be single-stranded, double-stranded, triple stranded, or a combination thereof. In some cases, an engineered nucleic acid may be single-stranded. In some cases, an engineered nucleic acid may be double-stranded. In some cases, an engineered nucleic acid may comprise single-stranded and double-stranded regions or portions.

In some cases, an engineered nucleic acid may be linear. In some cases, an engineered nucleic acid may be closed linear double-stranded (e.g., a doggybone DNA). In some cases, an engineered nucleic acid may be circular. In some cases, an engineered nucleic acid may be branched. An engineered nucleic acid may comprise a deoxyribonucleic acid (DNA) or ribonucleic acid (RNA). An engineered nucleic acid may comprise a peptide engineered nucleic acid (PNA), an unlocked engineered nucleic acid (UNA), a locked engineered nucleic acid (LNA), or a combination thereof. An engineered nucleic acid may comprise at least a coding sequence, at least a non-coding sequence, or a combination thereof. An engineered nucleic acid may comprise at least a coding sequence. An engineered nucleic acid may comprise at least a non-coding sequence. An engineered nucleic acid may comprise a coding or non-coding region of a gene or gene fragment, a locus defined from linkage analysis, an exons, an intron, an intein, or any combination thereof. A coding sequence may be a gene sequence, a codon-modified thereof, or a codon-optimized thereof. A coding sequence may be a variation of gene sequence. A non-coding sequence may be a gene sequence. A non-coding sequence may also be a variation of gene sequence. The variation may comprise a mutation. A mutation, in some cases, may comprise a nucleotide substitution, deletion, insertion, inversion, or a combination thereof. A mutation may alter the codon of a coding sequence. A mutation may not alter the codon of a coding sequence.

In some cases, an engineered nucleic acid may comprise a sequence of an animal gene or sequence. In some cases, an engineered nucleic acid may comprise a sequence of a non-animal gene or sequence. In some cases, a gene or sequence may comprise a gene or sequence of a cattle, equine, buffalo, pig, sheep, deer, chicken, duck, ostrich, turkey, pheasant, fish (e.g., swordfish, salmon, tuna, sea bass, trout, catfish, etc.), invertebrate (e.g., lobster, crab, shrimp, clams, oysters, mussels, sea urchin, etc.), reptile (e.g. snake, alligator, turtle, etc.), insect, amphibian (e.g. frog), or a combination thereof.

In some instances, an engineered nucleic acid may comprise an RNA. The RNA may comprise an engineered RNA. In some instances, an engineered RNA may comprise a messenger ribonucleic acid (mRNA), a micro ribonucleic acid (miRNA), a transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA (exRNA), a small cajal body-specific RNA (scaRNA), a silencing ribonucleic acid (siRNA), a short hairpin RNA (shRNA), a self-amplifying RNA (saRNA), a YRNA (small noncoding RNA), a heterogeneous nuclear RNA (HnRNA), an endless/circular RNA (eRNA), a trans-amplifying RNA (ta-RNA), a transfer RNA (tRNA), a ribosomal RNA, a short-hairpin RNA (shRNA), a ribozyme, a recombinant engineered RNA, a branched engineered RNA, a transacting RNA (tasiRNA), a cis-natural antisense transcript (cis-NAT), a vector, an isolated RNA, a combination thereof. The engineered RNA may comprise an mRNA, a miRNA, a tRNA, a siRNA, a saRNA, an eRNA, a ta-RNA, or a combination thereof. The engineered RNA may comprise an mRNA. The engineered RNA may comprise a miRNA. The engineered RNA may comprise a tRNA. The engineered RNA may comprise a siRNA. The engineered RNA may comprise a saRNA. The engineered RNA may comprise an eRNA. The engineered RNA may comprise a ta-RNA. The engineered RNA may comprise a tasiRNA. The engineered RNA may comprise a cis-NAT. The engineered RNA may comprise a viral sequence, a non-viral sequence, or a combination thereof. Using an RNA or an engineered RNA can have a beneficial advantage that the cell comprising the same needs not to be genetically modified. Thus, the cells or the derivatives thereof (for example, a meat product comprising the cells) can contain no genetic modifications.

In some instances, an engineered nucleic acid may comprise a cell type-specific gene sequence. A cell type specific-gene sequence may comprise the sequence of a gene that is expressed or specifically expressed of the cell type. The cell type may comprise an ectoderm, an endoderm, a mesoderm, or a combination thereof. The cell type may also comprise an adipogenic, angiogenic, cardiogenic, immunogenic, chondrogenic, endothelial, epithelial, hematopoietic, hepatogenic, myogenic, neurogenic, osteogenic, parenchymal, renal, retinal cell, or a combination thereof. In some cases, a cell may comprise a T cell, a B cell, a natural killer cell, a neutrophil, an eosinophil, a basophil, a mast cell, a monocyte, a macrophage, a dendritic cell, or a combination thereof. The cell type may also comprise a somatic cell, a stem cell, an immortalized cell, or a combination thereof. The stem cell or immortalized cell may comprise an induced pluripotent stem cell (iPSC), an embryonic stem cell (ESC), a mesenchymal stem cell (MSC), a satellite cell, a fibroblast, a cancer cell, or a combination thereof. In some instances, an engineered nucleic acid may comprise an adipogenic, angiogenic, cardiogenic, immunogenic, chondrogenic, endothelial, epithelial, hematopoietic, hepatogenic, myogenic, neurogenic, osteogenic, parenchymal, renal, retinal gene or sequence, or a combination thereof.

In some instances, an engineered nucleic acid may be monocistronic. In some cases, an engineered nucleic acid may be polycistronic. A monocistronic engineered nucleic acid may comprise one coding sequence. The coding sequence is configured to be recognized by a ribosome for translation. A polycistronic engineered nucleic acid may comprise at least two coding sequences. Each coding sequence is configured to be recognized by a ribosome for translation of the coding sequence. A polycistronic engineered nucleic acid may comprise at least about 2, 3, 4, 5, 6, 7, 8, 9, 10, 11, 12, 13, 14, 15, 16, 17, 18, 19, 20, 21, 22, 23, 24, 25, 50, 100 or more coding sequences. A polycistronic engineered nucleic acid may comprise at most about 2, 3, 4, 5, 6, 7, 8, 9, 10, 11, 12, 13, 14, 15, 16, 17, 18, 19, 20, 21, 22, 23, 24, 25, 50, or 100 coding sequences. An engineered nucleic acid may comprise at least about 2, 3, 4, 5, 6, 7, 8, 9, 10, 11, 12, 13, 14, 15, 16, 17, 18, 19, 20, 21, 22, 23, 24, 25, 50, 100 or more non-coding sequences. An engineered nucleic acid may comprise at most about 2, 3, 4, 5, 6, 7, 8, 9, 10, 11, 12, 13, 14, 15, 16, 17, 18, 19, 20, 21, 22, 23, 24, 25, 50, or 100 non-coding sequences. The coding sequences may be a same coding sequence (i.e., a copy of the same coding sequence). The coding sequences may be different coding sequences. The non-coding sequences may be a same non-coding sequence (i.e., a copy of the same non-coding sequence). The non-coding sequences may be different non-coding sequences.

In some instances, an engineered RNA may comprise at least a copy of a coding sequence. In some cases, an engineered RNA may comprise at least about 2, 3, 4, 5, 6, 7, 8, 9, 10, 11, 12, 13, 14, 15, 16, 17, 18, 19, 20, 21, 22, 23, 24, 25, 50, 100 or more copies of a coding sequence. In some cases, an engineered RNA may comprise at most about 2, 3, 4, 5, 6, 7, 8, 9, 10, 11, 12, 13, 14, 15, 16, 17, 18, 19, 20, 21, 22, 23, 24, 25, 50, or 100 copies of a coding sequence. In some instances, an engineered RNA may comprise at least a copy of a coding sequence. In some cases, an engineered RNA may comprise at least about 2, 3, 4, 5, 6, 7, 8, 9, 10, 11, 12, 13, 14, 15, 16, 17, 18, 19, 20, 21, 22, 23, 24, 25, 50, 100 or more copies of a non-coding sequence. In some cases, an engineered RNA may comprise at most about 2, 3, 4, 5, 6, 7, 8, 9, 10, 11, 12, 13, 14, 15, 16, 17, 18, 19, 20, 21, 22, 23, 24, 25, 50, or 100 copies of a non-coding sequence.

In some instances, an engineered RNA may comprise at least an RNA-regulatory element. In some cases, the RNA-regulatory element may be a non-coding sequence. In some instances, an engineered RNA may comprise at least about 1, 2, 3, 4, 5, 6, 7, 8, 9, 10, 11, 12, 13, 14, 15, 16, 17, 18, 19, 20, 21, 22, 23, 24, 25, 50, 100, 200 or more RNA-regulatory elements. In some instances, an engineered RNA may comprise at most about 1, 2, 3, 4, 5, 6, 7, 8, 9, 10, 11, 12, 13, 14, 15, 16, 17, 18, 19, 20, 21, 22, 23, 24, 25, 50, 100, or 200 RNA-regulatory elements. The RNA-regulatory elements may be a same RNA-regulatory element (i.e., a copy of the same RNA-regulatory element). The RNA-regulatory elements may be different RNA-regulatory elements. In some cases, an RNA-regulatory element may comprise a transcriptional regulatory element, a co-transcriptional regulatory element, a post-transcriptional regulatory element, a translational regulatory element, or any combination thereof. In some cases, an engineered nucleic acid may comprise a 5′-cap (or 5′cap), 5′-UTRs, a 3′-UTR, an alphavirus replication sequence, a Kozak sequence, a poly-A tail, a 5′cap, an internal Ribosome Entry Sequence (IRES), a 26S subgenomic promoter, an intron sequence, a stop codon sequence, a translation start site, an RNA-localization sequence, a sequence encoding a 2A self-cleaving peptide, or any combination thereof. In some cases, an engineered RNA may comprise an alphavirus replication sequence, a Kozak sequence, a poly-A tail, a 5′cap, an IRES, a 26S subgenomic promoter, or a combination thereof. An engineered nucleic acid may comprise an alphavirus replication sequence. An engineered nucleic acid may comprise a Kozak sequence. An engineered nucleic acid may comprise a poly-A tail. An engineered nucleic acid may comprise a 5′cap. An engineered nucleic acid may comprise an IRES. An engineered nucleic acid may comprise a 26S subgenomic promoter. An engineered nucleic acid may comprise a sequence encoding a 2A self-cleaving peptide. An alphavirus replication sequence may comprise a Venezuelan Equine Encephalitis alphavirus replication sequence.

In some instances, an engineered nucleic acid may be synthesized in vitro, ex vivo, in vivo, or a combination thereof. In some instances, an engineered nucleic acid may be synthesized in vitro. In some instances, an engineered nucleic acid may be synthesized ex vivo. In some instances, an engineered nucleic acid may be synthesized in vivo. In some instances, an engineered nucleic acid may be synthesized by in vitro transcription, in vitro synthesis, or a combination thereof. An engineered nucleic acid may be transcribed from a DNA template, a plasmid template, an RNA promoter, or a combination thereof. An engineered nucleic acid may be transcribed from a DNA template. An engineered nucleic acid may be transcribed from a plasmid template. An engineered nucleic acid may be transcribed from an RNA promoter. In some instances, the RNA promoter may comprise a T3 promoter, a T7 promoter, a SP6 promoter, or a combination thereof. The RNA promoter may comprise a T3 promoter. The RNA promoter may comprise a T7 promoter. The RNA promoter may comprise a SP6 promoter.

In some instances, an engineered nucleic acid may comprise at least a sequence encoding a cleavage site between two coding sequences. In some instances, an engineered nucleic acid may comprise at least about 2, 3, 4, 5 or more sequences encoding cleavage sites between two coding sequences. In some instances, an engineered nucleic acid may comprise at most about 2, 3, 4, or 5 sequences encoding cleavage sites between two coding sequences. The cleavage site may comprise a sequence encoding a 2A self-cleaving peptide. A 2A self-cleavage peptide may induce ribosomal skipping during translation of the two coding sequences. A 2A self-cleavage peptide may comprise T2A, P2A, E2A, F2A, or a combination thereof.

In some instances, the sequence of an engineered nucleic acid may be carried on a vector. A DNA sequence, counterpart, or complement thereof of an engineered RNA sequence may be carried on a vector. The vector may be transfected or transduced into a cell. The cell may use the vector or DNA to generate the engineered RNA, a sequence thereof, a derivative thereof, or a complement thereof. The vector may be used to generate an engineered RNA, a sequence thereof, a derivative thereof, or a complement thereof outside of a cell (such as by in vitro synthesis). The engineered RNA, sequence thereof, derivative thereof, or complement thereof can also be subsequently purified and transfected to another cell for growing using the method described herein (the cell generating the engineered RNA, sequence thereof, derivative thereof, or complement thereof and the cell being cultured or grown can be the same or different types of cell). Such methods can have a beneficial advantage of bypassing the synthesis or purification of non-nucleic acid molecules (such as peptides, polypeptides, or metabolites) which can be substantially more technical challenging and cost, relative to the synthesis or purification of a nucleic acid-based molecules.

In some instances, a nucleic acid may be modified. In some cases, a nucleic acid may be chemically modified. In some instances, chemically modified nucleic acid may reduce or inhibit degradation of the nucleic acid at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to a nucleic acid not chemically modified.

In some cases, a chemically modified nucleic acid may comprise an UNA, an LNA, or a combination thereof. In some instances, an LNA may have a structurally rigid modification. In some cases, an UNA may have a structurally flexible modification. In some cases, an UNA may comprise an acyclic analogue of RNA in which the bond between the C2′ and C3′ atoms of the ribose ring have been cleaved. In some cases, an LNA may comprise a modified nucleic acid in which the ribose moiety is modified with an extra bridge connecting the 2′ oxygen and 4′ carbon.

In some instances, an engineered nucleic acid may have a length of at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 200, 300, 400, 500, 600, 700, 800, 900, 1000, 5000, 10000, 50000, 100000, 500000 or more nucleotides. In some instances, an engineered nucleic acid may have a length of at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 200, 300, 400, 500, 600, 700, 800, 900, 1000, 5000, 10000, 50000, 100000, or 500000 nucleotides.

In some instances, the engineered nucleic acid, or a derivative thereof, may not be integrated into a genome of a cell. In some instances, the engineered nucleic acid, or a derivative thereof, may not be configured to be integrated into a genome of a cell. In some cases, the derivative of an engineered nucleic acid may be generated by the processing of a cell. In some cases, an engineered RNA may be amplified within a cell without being integrated into the genome of the cell. In some cases, an engineered RNA may be self-amplified within a cell without being integrated into the genome of the cell. In some instances, the engineered nucleic acid, or a derivative thereof, may also be integrated into a genome of a cell. In some instances, the engineered nucleic acid, or a derivative thereof, may be configured to be integrated into a genome of a cell.

In some instances, a vector harboring an engineered nucleic acid may have a length of at least about 100, 200, 300, 400, 500, 600, 700, 800, 900, 1000, 5000, 10000, 50000, 100000, 500000 or more nucleotides. In some instances, a vector harboring an engineered nucleic acid may have a length of at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 200, 300, 400, 500, 600, 700, 800, 900, 1000, 5000, 10000, 50000, 100000, or 500000 nucleotides.

### Polypeptides

In some instances, an engineered nucleic acid may comprise a sequence that encodes a polypeptide. In some instances, an engineered nucleic acid may encode a polypeptide. A polypeptide may comprise a cell proliferation regulator. A cell proliferation regulator may comprise a cellular component that can increase or decrease the cell proliferation of a cell. The cell proliferation may comprise at least a cell division, a cell growth, or a combination thereof. In some cases, the cell division may comprise an increase of a number of the cell. In some cases, the cell growth may comprise an increase of a mass of a cell. The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous cell proliferation regulator of a cell. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous cell proliferation regulator of a cell.

In some instances, a polypeptide may not have a sequence identity with respect to an endogenous cell proliferation regulator (e.g., the polypeptide may have 0% sequence identity with the endogenous cell proliferation regulator). In some cases, the polypeptide may fold into a structure that has conformational similarity to an endogenous cell proliferation regulator. The polypeptide may have a binding property or functional activity similar to that of the endogenous cell proliferation regulator.

In some instances, the binding properties or functional activities a polypeptide and an endogenous cell proliferation regulator may be measured using an experiment comprising assaying or measuring the binding property or functional activity of the polypeptide relative to the endogenous cell proliferation regulator. The experiment can comprise an in vitro or in vivo assay. For example, an experiment to assay for the binding properties of the polypeptide and the endogenous cell proliferation regulator may comprise a binding assay, wherein the polypeptide and the endogenous cell proliferation regulator are each assayed for binding the same binding target. Binding affinities of the binding target with the polypeptide and the endogenous cell proliferation regulator may be calculated. The polypeptide may have a binding property similar to that of the endogenous cell proliferation regulator if their binding affinities to the same binding target are within at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Similar assays may be used to measure the binding property of a polypeptide, a growth-factor, a growth-factor receptor, a cell cycle regulator, a cell proliferation signaling pathway regulator, a Maillard-reaction peptide, a metalloprotein and metallopeptide, or a combination thereof. In some cases, an experiment to assay the functional activities of the polypeptide and the endogenous cell proliferation regulator may comprise an assay that can measure or reflect the functional activities of the polypeptide and endogenous proliferation regulator. For example, an experiment to assay the functional actives of the polypeptide and the endogenous cell proliferation regulator may comprise an assay of the cell proliferation of a cell, biological or biochemical activity of a cell proliferation regulator, or a marker thereof. A parameter for the functional activities may be calculated from the assay. The polypeptide may have a functional activity similar to that of the endogenous cell proliferation regulator if their parameters of the functional activities measured using the assay are within at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Similar assays may be used to measure the functional activity of a polypeptide, a growth-factor, a growth-factor receptor, a cell cycle regulator, a cell proliferation signaling pathway regulator, a Maillard-reaction peptide, a metalloprotein and metallopeptide, or a combination thereof. In some instances, a polypeptide may comprise a variant of an endogenous cell proliferation regulator of a cell. The polypeptide may comprise a variation in the polypeptide sequence relative to a polypeptide sequence of an endogenous cell proliferation regulator of a cell. The variation may comprise a mutation. The variation may comprise an alteration of at least an amino acid residue of the endogenous cell proliferation regulator. For example, the variation may comprise altering an amino acid(s) of the polypeptide sequence of the endogenous cell proliferation regulator to a different amino acid(s). The variation may comprise removing an amino acid(s) of the polypeptide sequence of the endogenous cell proliferation regulator. The variation may also comprise an addition of an amino acid(s) to the polypeptide sequence of the endogenous cell proliferation regulator. In other cases, the variation may also comprise altering the polypeptide sequence of the endogenous cell proliferation regulator without a change in the composition of the amino acids in the polypeptide sequence of the endogenous cell proliferation regulator.

The variant of an endogenous cell proliferation regulator of a cell may have an altered activity relative to the endogenous cell proliferation regulator of the cell. The altered activity may comprise an alteration in a cell proliferation activity. In some cases, the variant of an endogenous cell proliferation regulator of a cell may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation activity, relative to the endogenous cell proliferation regulator of the cell. In some cases, the variant of an endogenous cell proliferation regulator of a cell may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation activity, relative to the endogenous cell proliferation regulator of the cell.

In some cases, the variant of an endogenous cell proliferation regulator may be an antagonistic form of the endogenous cell proliferation regulator. The endogenous cell proliferation regulator may be a negative cell proliferation regulator that decreases, limits, or inhibits cell proliferation when present in a cell. In some cases, a cell comprising the negative cell proliferation regulator may exhibit at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower cell proliferation or a marker thereof, relative to a cell without the negative cell proliferation regulator. In some cases, a cell comprising the negative cell proliferation regulator may exhibit at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 99% lower cell proliferation or a marker thereof, relative to a cell without the negative cell proliferation regulator. In some cases, the antagonistic form of an endogenous cell proliferation regulator may inhibit the cell proliferation-lowering activity of the negative cell proliferation regulator. For example, the cell comprising the negative cell proliferation regulator and the antagonistic form of an endogenous cell proliferation regulator may exhibit at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation or a marker thereof, relative to a cell with the negative cell proliferation regulator but without the antagonistic form of the endogenous cell proliferation regulator. The cell comprising the negative cell proliferation regulator and the antagonistic form of the endogenous cell proliferation regulator may exhibit at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation or a marker thereof, relative to a cell with the negative cell proliferation regulator but without the antagonistic form of the endogenous cell proliferation regulator. The antagonistic form of an endogenous cell proliferation regulator may be a dominant negative mutant of the endogenous cell proliferation regulator.

In other cases, the cell proliferation activity may be measured by a biological or biochemical activity of a cell proliferation marker. A cell proliferation marker may comprise a component that is involved in the biological or biochemical activity of the cell proliferation pathway of the cell or reflect the activities thereof. An alteration of the cell proliferation pathway of the cell may increase or decrease the biological or biochemical activity of the cell proliferation marker. In some cases, the cell proliferation marker may comprise an endogenous component involved in the cell proliferation pathway of the cell. In other cases, the cell proliferation marker may comprise an exogenous molecule that can reflect or report the cell proliferation pathway of the cell. In some cases, the variant of an endogenous cell proliferation regulator of a cell may exhibit at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation marker activity, relative to the endogenous cell proliferation regulator of the cell. In some cases, the variant of an endogenous cell proliferation regulator of a cell may exhibit at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation marker activity, relative to the endogenous cell proliferation regulator of the cell.

In other cases, the cell proliferation marker may comprise an exogenous molecule that can reflect or report the cell proliferation pathway of the cell.

In some cases, the variant of an endogenous cell proliferation regulator of a cell may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower cell proliferation marker activity, relative to the endogenous cell proliferation regulator of the cell. In some cases, the variant of an endogenous cell proliferation regulator of a cell may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower cell proliferation marker activity, relative to the endogenous cell proliferation regulator of the cell.

When comparing the cell proliferation activity or cell proliferation marker activity induced by an endogenous cell proliferation regulator, an experiment may comprise assaying or measuring the cell proliferation or cell proliferation marker of two cells, wherein one cell comprises, is contacted with, or expresses the endogenous cell proliferation regulator, or an engineered nucleic acid encoding thereof, wherein another cell does not comprise, is not contacted with, or does not express the variant of the endogenous cell proliferation regulator, or an engineered nucleic acid encoding thereof.

When comparing the cell proliferation activity or cell proliferation marker activity induced by a variant of an endogenous cell proliferation regulator relative to the endogenous cell proliferation regulator, an experiment may comprise assaying or measuring the cell proliferation or cell proliferation marker of two cells, wherein one cell comprises, is contacted with, or expresses the variant of the endogenous cell proliferation regulator, or an engineered nucleic acid encoding thereof, wherein another cell comprises, is contacted with, or expresses the endogenous cell proliferation regulator, or an engineered nucleic acid encoding thereof.

When comparing the cell proliferation activity or cell proliferation marker activity induced by an exogenous cell proliferation regulator, an experiment may comprise assaying or measuring the cell proliferation or cell proliferation marker of two cells, wherein one cell comprises, is contacted with, or expresses the exogenous cell proliferation regulator, or an engineered nucleic acid encoding thereof, wherein another cell does not comprise, is not contacted with, or does not express the exogenous cell proliferation regulator, or an engineered nucleic acid encoding thereof.

In some cases, a polypeptide may comprise a growth-factor, a growth-factor receptor, a cell proliferation signaling pathway regulator, a cell-cycle regulator, an immune response regulator, a Maillard reaction peptide, a metallopeptide, or a combination thereof. A polypeptide may comprise a growth-factor. A polypeptide may comprise a growth-factor receptor. A polypeptide may comprise a cell proliferation signaling pathway regulator. A polypeptide may comprise a cell-cycle regulator. A polypeptide may comprise a Maillard reaction peptide. A polypeptide may comprise a metallopeptide. A polypeptide may comprise an immune response regulator. For example, an immune response regulator may increase or decrease the activity of an immune response pathway of a cell or organism, when present in the cell or organism.

In some cases, the polypeptide may comprise Basic Fibroblast Growth-factor (FGF), FGF-2, FGF-7, Soluble IFN alpha/beta receptor B18 (B18R), Transforming Growth-factor Beta (TGF-beta), TGF-beta 1, Neuregulin 1 (NRG1), Heregulin, Insulin, Insulin-like Growth-factor, IGF-1, Transferrin, Epidermal Growth-factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-derived growth factor (PDGF), PDGF-BB, vascular endothelial growth factor (VEGF), VEGF-A, Hepatocyte growth factor (HGF), bovine serum albumin, immune evasion peptide E3, immune evasion peptide K3, any functional variants thereof, or a combination thereof. The polypeptide may comprise FGF or a functional variant thereof. The polypeptide may comprise TGF-beta or a functional variant thereof. The polypeptide may comprise NRG1 or a functional variant thereof. The polypeptide may comprise Heregulin or a functional variant thereof. The polypeptide may comprise Insulin or a functional variant thereof. The polypeptide may comprise Insulin-like Growth-factor or a functional variant thereof. The polypeptide may comprise Transferrin or a functional variant thereof. The polypeptide may comprise EGF or a functional variant thereof. The polypeptide may comprise Activin or a functional variant thereof. The polypeptide may comprise Nodal or a functional variant thereof. The polypeptide may comprise Wnt or a functional variant thereof. The polypeptide may comprise Albumin or a functional variant thereof. The polypeptide may comprise myoglobin or a functional variant thereof. The polypeptide may comprise Haemoglobin or a functional variant thereof. The polypeptide may comprise FGF-2 or a functional variant thereof. The polypeptide may comprise FGF-7 or a functional variant thereof. The polypeptide may comprise B18R or a functional variant thereof. The polypeptide may comprise TGF-beta 1 or a functional variant thereof. The polypeptide may comprise IGF-1 or a functional variant thereof. The polypeptide may comprise Leukaemia Inhibitory Factor or a functional variant thereof. The polypeptide may comprise BMP-2 or a functional variant thereof. The polypeptide may comprise BMP-7 or a functional variant thereof. The polypeptide may comprise PDGF or a functional variant thereof. The polypeptide may comprise PDGF-BB or a functional variant thereof. The polypeptide may comprise VEGF or a functional variant thereof. The polypeptide may comprise VEGF-A or a functional variant thereof. The polypeptide may comprise HGF or a functional variant thereof. The polypeptide may comprise bovine serum albumin or a functional variant thereof. The polypeptide may comprise immune evasion peptide E3 or a functional variant thereof. The polypeptide may comprise immune evasion peptide K3 or a functional variant thereof. In some instances, a functional variant of a polypeptide may have a functional activity similar to that of any one of FGF, FGF-2, FGF-7, B18R, TGF-beta, TGF-beta 1, NRG1, Heregulin, Insulin, Insulin-like Growth-factor, IGF-1, Transferrin, EGF, Activin, Nodal, Wnt, Albumin, myoglobin, Inhibitory Factor, BMP, BMP-2, BMP-7, PDGF, PDGF-BB, VEGF, VEGF-A, HGF, bovine serum albumin, immune evasion peptide E3, immune evasion peptide K3, or a combination thereof. An experiment to measure the functional activity may comprise an assay of the cell proliferation of a cell, biological or biochemical activity of the polypeptide and the functional variant of the polypeptide, or a marker thereof. A parameter for the functional activities may be calculated from the assay. The functional variant of a polypeptide may have a functional activity similar to that of the polypeptide if their parameters of the functional activities measured using the assay are within at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Similar assays may be used to measure the functional activities of any polypeptides described elsewhere in this disclosure.

The polypeptide may comprise a mammalian polypeptide or polypeptide sequence. The polypeptide may comprise a non-mammalian polypeptide or polypeptide sequence. The polypeptide may comprise a polypeptide or polypeptide sequence from fish, insect, reptile, bird, amphibian, plant, yeast, bacteria, or any combination thereof.

### Growth-Factors

A growth-factor may also comprise a molecule that can increase, stimulate, or activate cell proliferation, cell migration, cell clustering, cell differentiation, or a combination thereof of a cell. A growth-factor may comprise a molecule that can increase, stimulate, or activate a cell proliferation. A growth-factor may comprise a molecule that can increase, stimulate, or activate a cell division. A growth-factor may comprise a molecule that can increase, stimulate, or activate a cell growth. A growth-factor may be secreted. A growth-factor may not be secreted. A growth-factor may comprise an extracellular protein. A growth-factor may comprise an intracellular protein. A growth-factor may comprise a membrane protein. A growth-factor may comprise a plasma membrane protein. A growth-factor that is a plasma membrane protein may comprise an intracellular domain, an extracellular domain, or a combination thereof.

A polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a growth-factor of a cell. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a growth-factor of a cell. The growth-factor may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous growth-factor of a cell. The growth-factor may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous growth-factor of a cell. The growth-factor may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an exogenous growth-factor of a cell. The growth-factor may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an exogenous growth-factor of a cell.

In some instances, a growth-factor may not have a sequence identity with respect to an endogenous growth-factor (e.g., the growth-factor may have 0% sequence identity with the endogenous growth-factor). In some cases, the growth-factor may fold into a structure that has conformational similarity to an endogenous growth-factor. The growth-factor may have a binding property or functional activity similar to that of the endogenous growth-factor.

In some instances, a polypeptide may comprise a variant of a growth-factor. In some cases, the variant of a growth-factor may be an active form of the growth-factor. In some cases, the variant of a growth-factor may be an inactive form of the growth-factor. In some cases, the variant of the growth-factor (e.g., active or inactive form of the growth-factor) may increase, stimulate, or activate cell proliferation of a cell when present in an amount that is insufficient for the non-variant form of the growth-factor to increase, stimulate, or activate cell proliferation of the cell.

In some cases, the variant of a growth-factor may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation activity, relative to the growth-factor that is not in the variant form. In some cases the variant of a growth-factor may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation activity, relative to the growth-factor that is not in the variant form. In some cases, the cell proliferation activity of the growth-factor may be measured by the methods described elsewhere in this disclosure.

In some cases, the variant of a growth-factor of a cell may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower cell proliferation activity, relative to the growth-factor. In some cases, the variant of a growth-factor may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 99% lower cell proliferation activity, relative to the growth-factor. In some cases, the variant of a growth-factor may be an antagonistic form of a growth-factor. The variant of the growth-factor may be a dominant negative mutant of the growth-factor. In some cases, a variant of a growth-factor may comprise a receptor ligand. A receptor ligand may comprise a modulator or allosteric modulator. In some cases, the modulator or allosteric modulator may comprise a co-agonist, enhancer, allosteric enhancer, agonist, allosteric agonist, ago-allosteric modulator, or a combination thereof of a growth-factor.

In some cases, a growth-factor may comprise a peptide or polypeptide. In some cases, a growth-factor may be a peptide or polypeptide. In other cases, a growth-factor may not comprise a peptide or polypeptide. The growth-factor may not be amino acid based, peptide-based, polypeptide-based, or a combination thereof. In some cases, a growth-factor may be a metabolite that is not amino acid based or does not comprise an amino acid. In some cases, a growth-factor may comprise an organic or inorganic compound. In some cases, a growth-factor may comprise a saccharide. In some cases, a growth-factor may comprise a lipid. In some cases, a growth-factor may not be encoded by an engineered nucleic acid. In some instances, a polypeptide encoded by an engineered nucleic acid may facilitate the synthesis of the growth-factor. For example, the polypeptide encoded by the engineered nucleic acid may comprise an enzyme or signaling pathway component that is involved in the synthesis of the growth-factor.

### Growth-Factor Receptors

In some instances, a polypeptide may comprise a growth-factor receptor. A growth-factor receptor may bind a growth-factor. A growth-factor receptor may bind an exogenous growth-factor, an endogenous growth-factor, or a combination thereof. A growth-factor receptor may increase, stimulate, or activate cell proliferation, cell migration, cell clustering, cell differentiation, or a combination thereof of a cell. A growth-factor receptor may increase, stimulate, or activate a cell proliferation. A growth-factor receptor may increase, stimulate, or activate a cell division. A growth-factor receptor may increase, stimulate, or activate a cell growth. Binding of the growth-factor receptor of a cell with the growth-factor may increase, stimulate, or activate the proliferation of the cell.

A growth-factor receptor may not be secreted. A growth-factor receptor may comprise an extracellular protein. A growth-factor receptor may comprise an intracellular protein. A growth-factor receptor may comprise a membrane protein. A growth-factor receptor may comprise a plasma membrane protein. A growth-factor receptor that is a plasma membrane protein may comprise an intracellular domain, an extracellular domain, or a combination thereof. The binding of the growth-factor may increase, stimulate, or activate the activity of a growth-factor receptor to increase or stimulate the cell proliferation of the cell. The binding of the growth-factor may also decrease, inactivate, or inhibit the activity of a growth-factor receptor to increase or stimulate the cell proliferation of the cell.

The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a growth-factor receptor. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a growth-factor receptor. The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous growth-factor receptor of a cell. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an endogenous growth-factor receptor of a cell. The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an exogenous growth-factor receptor of a cell. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to an exogenous growth-factor receptor of a cell.

In some instances, a growth-factor receptor may not have a sequence identity with respect to an endogenous growth-factor receptor (e.g., the growth-factor receptor may have 0% sequence identity with the endogenous growth-factor receptor). In some cases, the growth-factor receptor may fold into a structure that has conformational similarity to an endogenous growth-factor receptor. The growth-factor receptor may have a binding property or functional activity similar to that of the endogenous growth-factor receptor.

In some instances, a polypeptide may comprise a variant of a growth-factor receptor. In some cases, the variant of a growth-factor receptor may be an active form of the growth-factor receptor. In some cases, the active form of the growth-factor receptor may increase, stimulate, or activate cell proliferation of a cell in a limited amount of the growth-factor that can bind to and/or activate the growth-factor receptor that is not in the active form. In some cases, the active form of the growth-factor receptor may increase, stimulate, or activate cell proliferation of a cell in an absence of the growth-factor that can bind to and/or activate the growth-factor receptor that is not in the active form. The variation of the growth-factor receptor may comprise any variation described elsewhere in this disclosure.

In some cases, the variant of a growth-factor receptor may be an inactive form of the growth-factor receptor. In some cases, the inactive form of the growth-factor receptor may increase, stimulate, or activate cell proliferation of a cell in a limited amount of the growth-factor that can bind to and/or activate the growth-factor receptor that is not in the inactive form. In some cases, the inactive form of the growth-factor receptor may increase, stimulate, or activate cell proliferation of a cell in an absence of the growth-factor that can bind to and/or activate the growth-factor receptor that is not in the inactive form. In such cases, the growth-factor receptor not in the inactive form may inhibit, inactivate, or eliminate cell proliferation. Hence, inactivation of such growth-factor receptor may relieve the inhibition, inactivation, or elimination of the cell proliferation.

In some cases, the variant of a growth-factor receptor may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation activity, relative to the growth-factor receptor that is not in the variant form. In some cases the variant of a growth-factor receptor may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation activity, relative to the growth-factor receptor that is not in the variant form. In some cases, the cell proliferation activity of the growth-factor receptor may be measured by the methods described elsewhere in this disclosure.

### Cell-Cycle Regulators

In some instances, a polypeptide may comprise a cell-cycle regulator. A cell-cycle regulator may comprise a component that activates or inhibits the progression of the cell-cycling of a cell. A cell-cycle may comprise an increase in size (or mass) phase (gap 1 or G1), a DNA synthesis phase (synthesis or S phase), a preparation to divide phase (gap 2 or G2) phase, and a division phase (mitosis or M phase). A cell-cycle regulator may activate, stimulate, elongate/prolong any one or combination of the cell-cycle phases. A cell-cycle regulator may also inactivate, eliminate, shorten any one or combination of the cell-cycle phases. A cell-cycle regulator may increase, stimulate, or activate cell proliferation, cell migration, cell clustering, cell differentiation, or a combination thereof of a cell.

The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a cell-cycle regulator. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a cell-cycle regulator. The cell-cycle regulator may be an endogenous cell-cycle regulator. The cell-cycle regulator may be an exogenous cell-cycle regulator.

In some instances, a cell-cycle regulator may not have a sequence identity with respect to an endogenous cell-cycle regulator (e.g., the cell-cycle regulator may have 0% sequence identity with the endogenous cell-cycle regulator). In some cases, the cell-cycle regulator may fold into a structure that has conformational similarity to an endogenous cell-cycle regulator. The cell-cycle regulator may have a binding property or functional activity similar to that of the endogenous cell-cycle regulator.

In some instances, a polypeptide may comprise a variant of a cell-cycle regulator. In some cases, the variant of a cell-cycle regulator may be an active form of the cell-cycle regulator. In some cases, the active form of the cell-cycle regulator may increase, stimulate, or activate cell proliferation of a cell in a limited amount of the growth-factor that can bind to and/or activate the cell-cycle regulator that is not in the active form. In some cases, the active form of the cell-cycle regulator may increase, stimulate, or activate cell proliferation of a cell in an absence of the growth-factor that can bind to and/or activate the cell-cycle regulator that is not in the active form. The variation of the cell-cycle regulator may comprise any variation described elsewhere in this disclosure.

In some cases, the variant of a cell-cycle regulator may be an inactive form of the cell-cycle regulator. In some cases, the inactive form of the cell-cycle regulator may increase, stimulate, or activate cell proliferation of a cell in a limited amount of the growth-factor that can bind to and/or activate the cell-cycle regulator that is not in the inactive form. In some cases, the inactive form of the cell-cycle regulator may increase, stimulate, or activate cell proliferation of a cell in an absence of the growth-factor that can bind to and/or activate the cell-cycle regulator that is not in the inactive form. In such cases, the cell-cycle regulator not in the inactive form may inhibit, inactivate, or eliminate cell proliferation. Hence, inactivation of such cell-cycle regulator may relieve the inhibition, inactivation, or elimination of the cell proliferation.

In some cases, the variant of a cell-cycle regulator may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation activity, relative to the cell-cycle regulator that is not in the variant form. In some cases the variant of a cell-cycle regulator may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation activity, relative to the cell-cycle regulator that is not in the variant form. In some cases, the cell proliferation activity of the cell-cycle regulator may be measured by the methods described elsewhere in this disclosure.

### Cell Proliferation Signaling Pathway Regulators

In some instances, a polypeptide may comprise a cell proliferation signaling pathway regulator. A cell proliferation signaling pathway regulator may comprise a component that is not a growth-factor, a growth-factor receptor, or a cell-cycle regulator but otherwise activates or inhibits the cell proliferation. Examples of a cell proliferation signaling pathway regulator may comprise a signaling cascade of a cell proliferation pathway, a scaffold protein that is involved in the signaling of a cell proliferation pathway, protein turnover regulators, transcriptional regulators, translational regulators, transcriptional machinery, translational machinery, post-transcriptional regulators, post-translational regulators, enzymes for nucleic acid synthesis, enzymes for metabolite synthesis, RNA-binding protein, or a combination thereof. A cell proliferation signaling pathway regulator may increase, stimulate, or activate cell proliferation, cell migration, cell clustering, cell differentiation, or a combination thereof of a cell.

The polypeptide may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a cell proliferation signaling pathway regulator. The polypeptide may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a cell proliferation signaling pathway regulator. The cell proliferation signaling pathway regulator may be an endogenous cell proliferation signaling pathway regulator. The cell proliferation signaling pathway regulator may be an exogenous cell proliferation signaling pathway regulator.

In some instances, a cell proliferation signaling pathway regulator may not have a sequence identity with respect to an endogenous cell proliferation signaling pathway regulator (e.g., the cell proliferation signaling pathway regulator may have 0% sequence identity with the endogenous cell proliferation signaling pathway regulator). In some cases, the cell proliferation signaling pathway regulator may fold into a structure that has conformational similarity to an endogenous cell proliferation signaling pathway regulator. The cell proliferation signaling pathway regulator may have a binding property or functional activity similar to that of the endogenous cell proliferation signaling pathway regulator.

In some instances, a polypeptide may comprise a variant of a cell proliferation signaling pathway regulator. In some cases, the variant of a cell proliferation signaling pathway regulator may be an active form of the cell proliferation signaling pathway regulator. In some cases, the active form of the cell proliferation signaling pathway regulator may increase, stimulate, or activate cell proliferation of a cell in a limited amount of the growth-factor that can bind to and/or activate the cell proliferation signaling pathway regulator that is not in the active form. In some cases, the active form of the cell proliferation signaling pathway regulator may increase, stimulate, or activate cell proliferation of a cell in an absence of the growth-factor that can bind to and/or activate the cell proliferation signaling pathway regulator that is not in the active form. The variation of the cell proliferation signaling pathway regulator may comprise any variation described elsewhere in this disclosure.

In some cases, the variant of a cell proliferation signaling pathway regulator may be an inactive form of the cell proliferation signaling pathway regulator. In some cases, the inactive form of the cell proliferation signaling pathway regulator may increase, stimulate, or activate cell proliferation of a cell in a limited amount of the growth-factor that can bind to and/or activate the cell proliferation signaling pathway regulator that is not in the inactive form. In some cases, the inactive form of the cell proliferation signaling pathway regulator may increase, stimulate, or activate cell proliferation of a cell in an absence of the growth-factor that can bind to and/or activate the cell proliferation signaling pathway regulator that is not in the inactive form. In such cases, the cell proliferation signaling pathway regulator not in the inactive form may inhibit, inactivate, or eliminate cell proliferation. Hence, inactivation of such cell proliferation signaling pathway regulator may relieve the inhibition, inactivation, or elimination of the cell proliferation.

In some cases, the variant of a cell proliferation signaling pathway regulator may have at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold or higher cell proliferation activity, relative to the cell proliferation signaling pathway regulator that is not in the variant form. In some cases the variant of a cell proliferation signaling pathway regulator may have at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, or 1000-fold higher cell proliferation activity, relative to the cell proliferation signaling pathway regulator that is not in the variant form. In some cases, the cell proliferation activity of the cell proliferation signaling pathway regulator may be measured by the methods described elsewhere in this disclosure.

### Maillard-Reaction Peptides

In some instances, a polypeptide may comprise a Maillard-reaction peptide. A Maillard-reaction polypeptide may facilitate a Maillard-reaction of the polypeptide. In some cases, a Maillard-reaction polypeptide present within a cell may facilitate a Maillard-reaction of other molecules of the cell. The molecules may comprise an amino acid, peptide, polypeptide, protein, carbohydrate, or lipid of a cell. A cell or a product generated by the cell (e.g., a meat product) comprising a Maillard peptide may have a characteristic of a cell or product that has undergone the Maillard reaction. For example, the meat product may have a taste or texture profile substantially similar to a meat product that has undergone the Maillard reaction.

In some instances, a Maillard-reaction or Maillard reaction may comprise a plurality of reactions. In some cases, may comprise the plurality of reactions may comprise at least a non-enzymatic reaction. In other cases, the plurality of reactions may comprise a plurality of non-enzymatic reactions. In other cases, a Maillard reaction may also be referred to as “browning.”

In some cases, a Maillard reaction may occur at a temperature that is at least about 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., 178° C., 179° C., 180° C., 181° C., 182° C., 183° C., 184° C., 185° C. or higher. In some cases, a Maillard reaction may occur at a temperature that is at most about 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., 178° C., 179° C., 180° C., 181° C., 182° C., 183° C., 184° C., or 185° C.

The plurality of reactions may comprise glycosylation, multiplication, and polymerization. In some cases, the Maillard reaction may comprise a carbohydrate. The carbohydrate may comprise a reducing carbohydrate. The reducing carbohydrate may comprise a reducing sugar. In some cases, a Maillard reaction may comprise a carbohydrate and an amino compound. The amino compound may comprise an amine. In some cases, a Maillard reaction may comprise generating a glycosylamine from the carbohydrate and the amino compound. In some cases, a Maillard reaction may also comprise generating a glycosylamine from a reducing sugar and an amine. In some cases, the glycosylamine, the reducing sugar, the amine, or any combination thereof, may be converted to an amino deoxy fructose. The conversion may comprise the glycosylation. The glycosylation may comprise Amadori rearrangement. For compounds in a meat product to have undergone Amadori rearrangements, the meat products may be emulsified, foamed, solubilized, gelled, heat-stabilized, or any combination thereof. The reactive carbonyl group of the sugar may react with the nucleophilic amino group of the amino acid and form a complex mixture of poorly defined molecules. These molecules may provide a plurality of aromas and flavors to a meat product. In some cases, the Maillard reaction may comprise multiplication of these poorly defined compounds. In some cases, the multiplication of the poorly defined compounds may comprise fissions, dehydrations, Strecker degradations of the compounds. In some cases, these poorly defined compounds may be converted to advanced glycation end products. These advanced glycation end products may be antimicrobial, antioxidative, anticarcinogenic, antimutagenic, or any combinations thereof. In other cases, the converted/multiplicated poorly defined compounds may be polymerized. The amino acid groups of the converted/multiplicated poorly defined compounds may be oxidated, condensed, cyclized, rearranged, or any combinations thereof. The polymerization may generate melanoidins.

A Maillard-reaction peptide comprises an amino acid-rich polypeptide. For a polypeptide that is amino acid rich for one specific amino acid, at least about 5%, 10%, 15%, 20%, 25%, 30%, 35%, 40%, 45%, 50%, 55%, 60%, 65%, 70%, 75%, 80%, 85%, 90%, 95%, or 100% of all amino acids of the polypeptide may comprise the one specific amino acid. For a polypeptide that is amino acid rich for one specific amino acid, at most about 5%, 10%, 15%, 20%, 25%, 30%, 35%, 40%, 45%, 50%, 55%, 60%, 65%, 70%, 75%, 80%, 85%, 90%, 95%, or 100% of all amino acids of the polypeptide may also comprise the one specific amino acid. The amino acid may comprise proteinogenic amino acid, non-proteinogenic amino acid, or a combination thereof. The amino acids may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a combination thereof.

A Maillard-reaction peptide comprises a lysine-rich polypeptide, a cysteine-rich polypeptide, an arginine-rich polypeptide, or a combination thereof. A Maillard-reaction peptide comprises a lysine-rich polypeptide. A Maillard-reaction peptide comprises a cysteine-rich polypeptide. A Maillard-reaction peptide comprises an arginine-rich polypeptide.

### Metalloproteins and Metallopeptides

In some instances, a polypeptide may comprise a metalloprotein. A metalloprotein may comprise a polypeptide that binds to at least a metal ion. The metalloprotein, in a secondary-folded from, tertiary-folded form, or a combination thereof, may bind to a metal ion. The metal ion may comprise calcium, potassium, cobalt, nickel, copper, zinc, or a combination thereof.

A polypeptide may comprise a metallopeptide. In some instances, a metallopeptide may comprise a peptide sequence or fragment of a metalloprotein. In some cases, a metallopeptide may be a mimetic of a metalloprotein. The metallopeptide may comprise an amino acid sequence, when translated or synthesized alone, that can bind to a metal ion that can be bound by the metalloprotein. A metallopeptide may comprise at most about 5%, 10%, 15%, 20%, 25%, 30%, 35%, 40%, 45%, 50%, 55%, 60%, 65%, 70%, 75%, 80%, 85%, 90%, 95%, 99% or more (but less than 100%) of the amino acid residue sequences of the metalloprotein.

In some cases, the metal ion may facilitate a chemical reaction of a cell. In some cases, the chemical reaction may comprise the Maillard reaction as described herein. In some cases, a cell comprising a comprising the metalloprotein or metallopeptide may comprise a higher amount of the metal ion (by binding to the metalloprotein or metallopeptide), relative to that of a cell that does not comprise the metalloprotein or metallopeptide. In some cases, a cell comprising a comprising the metalloprotein or metallopeptide may have a higher tendency to undergo Maillard reaction, relative to that of a cell that does not comprise the metalloprotein or metallopeptide. A cell or a product generated by the cell (e.g., a meat product) comprising the metalloprotein or metallopeptide) may have a characteristic of a cell or product that has undergone the Maillard reaction.

### Therapeutic Products

In some instances, a polypeptide may encode a therapeutic product. In other cases, a polypeptide may facilitate a synthesis of a therapeutic product.

In some cases, a therapeutic product may have a therapeutic effect when administered. A therapeutic effect may comprise an inhibition, amelioration, mitigation, treatment, and/or prevention of a disease condition. In some cases, a therapeutic product may comprise a cell used in cell therapy. In some cases, a therapeutic product may also comprise a cell, a vaccine, an organ, a product produced by the cell. The product produced by the cell may comprise a polypeptide, a nucleic acid molecule, a chemical compound, or a combination thereof. In some cases, a polypeptide may comprise a protein. A protein may comprise an antibody, an enzyme, a signaling molecule, an enzyme inhibitor, a hormone, a cytokine, a growth-factor or a combination thereof. A nucleic acid molecule used as a therapeutic product may comprise any nucleic molecules described elsewhere in this disclosure. In some cases, a chemical compound may comprise an organic or inorganic compound. In some cases, a compound may comprise a nutrient. In some cases, a cell used as a therapeutic product may comprise any cell described elsewhere in this disclosure. In some cases, the therapeutic product may comprise a human or a non-human cell, protein, nucleic acid molecule, chemical compound, or a combination thereof. In some cases, a product produced by the cell may comprise an antimicrobial molecule. The antimicrobial molecule may be an antibacterial, an antifungal, or an antiparasitic molecule.

A therapeutic product may comprise a medicine. A medicine may comprise a chemical or a collection and/or mixture of chemicals that has a therapeutic effect when administered. In some cases, a therapeutic product may comprise a drug product. In some cases, a drug product may be a therapeutic product in a dosage form. In some cases, a therapeutic product may comprise a pharmaceutically active ingredient. A pharmaceutically active ingredient may comprise a chemical and/or cell that has a direct pharmaceutical activity contributes to and/or is responsible for the inhibition, amelioration, mitigation, treatment, and/or prevention of a disease condition. In some cases, a therapeutic product may comprise a drug substance. In some cases, a drug substance may comprise a pure form of the pharmaceutically active ingredient. In some cases, a sample comprising a drug substance may comprise at least 50%, 60%, 70%, 80%, 90%, or 100%, by volume, weight, and/or number of molecules, of the drug substance in the sample.

### Culture Medium

Provided herein are culture media. A culture medium may comprise at least a molecule for supporting growth of a cell. A culture medium may comprise at least a molecule for supporting growth of a cell in vitro or ex vivo. A culture medium may comprise a carbon source, a nitrogen source, a mineral source, a vitamin source, water, salt, oxygen, carbon dioxide, or a combination thereof.

A culture medium may not comprise a polypeptide. A culture medium may comprise a limited amount of a polypeptide. The polypeptide may comprise an exogenous polypeptide. The polypeptide may comprise any polypeptides described elsewhere in this disclosure. The polypeptide may be synthesized or derived in vivo, in vitro, ex vivo, or a combination thereof. In some cases, a culture medium may comprise a limited amount of polypeptide or exogenous polypeptide. The culture medium without a polypeptide or with a limited amount of polypeptide may not be sufficient for a cell to proliferate within the culture medium. The culture medium without a polypeptide or with a limited amount of polypeptide may not be sufficient for a cell without an engineered nucleic acid to proliferate within the culture medium. In some cases, the culture medium without a polypeptide or with a limited amount of polypeptide may be sufficient for a cell comprising an engineered nucleic acid to proliferate within the culture medium. In some cases, the cell may also be an engineered cell. The polypeptide or exogenous polypeptide present within the culture medium may comprise any polypeptides encoded by the engineered nucleic acid described elsewhere in this disclosure. In some cases, the polypeptide or exogenous polypeptide present within the culture medium in a limited amount or not present within the culture medium may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81 82%, 83%, 84%, 85%, 86%, 87%, 88%, 89%, 90%, 91%, 92%, 93%, 94%, 95%, 96%, 97%, 98%, 99%, or 100% sequence identity to a polypeptide encoded by an engineered nucleic acid. The polypeptide or exogenous polypeptide present within the culture medium in a limited amount or not present within the culture medium may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide encoded by an engineered nucleic acid. In some cases, the polypeptide or exogenous polypeptide present within the culture medium in a limited amount or not present within the culture medium may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide derived from a cell. The polypeptide or exogenous polypeptide present within the culture medium in a limited amount or not present within the culture medium may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide derived from a cell. The cell may be engineered. The cell may not be engineered. The polypeptide that is a growth-factor may also be referred to as a peptide.

A culture medium may not comprise a growth-factor. The growth-factor may comprise an exogenous growth-factor. In some cases, a culture medium may comprise a limited amount of growth-factor or exogenous growth-factor. The culture medium without a growth-factor or with a limited amount of growth-factor may not be sufficient for a cell to proliferate within the culture medium. In some cases, the cell may not be an engineered cell. The culture medium without a growth-factor or with a limited amount of growth-factor may not be sufficient for a cell without an engineered nucleic acid to proliferate within the culture medium. In some cases, the culture medium without a growth-factor or with a limited amount of growth-factor may not be sufficient for a cell comprising an engineered nucleic acid to proliferate within the culture medium. The growth-factor or exogenous growth-factor present within the culture medium may comprise any polypeptides encoded by the engineered nucleic acid described elsewhere in this disclosure. In some cases, the growth-factor or exogenous growth-factor present within the culture medium in a limited amount or not present within the culture medium may have at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide encoded by an engineered nucleic acid. The growth-factor or exogenous growth-factor present within the culture medium in a limited amount or not present within the culture medium may have at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide encoded by an engineered nucleic acid.

In some cases, a culture medium may comprise a limited amount of growth-factor. In some cases, a culture medium may comprise a limited amount of exogenous growth-factor. In some cases, a culture medium may comprise a limited amount of endogenous growth-factor. In other cases, a culture medium may not comprise a growth-factor (endogenous or exogenous). The limited amount of growth factors (endogenous or exogenous) may comprise any amounts described herein. For example, in some cases, a culture medium may comprise at most about 100 milligrams per liter (mg/L), 10 mg/L, 1 mg/L, 100 micrograms per liter (μg/L), 10 μg/L, 1 μg/L, 100 nanograms per liter (ng/L), 10 ng/L, 1 ng/L, 100 picograms per liter (pg/L), 10 pg/L, 1 pg/L, 100 femtograms/L (fg/L), 10 fg/L, 1 fg/L or less growth factor. In some cases, a culture medium may comprise at most about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L growth factor. In some cases, a culture medium may comprise at most about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, 1 fg/L or less exogenous growth factor. In some cases, a culture medium may comprise at least about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L exogenous growth factor. In some cases, a culture medium may comprise at most about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, 1 fg/L or less endogenous growth factor. In some cases, a culture medium may comprise at least about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L endogenous growth factor. In some cases, a culture medium may comprise at most about 100 millimolar (mM), 10 mM, 1 mM, 100 micromolar (μM), 10 μM, 1 μM, 100 nanomolar (nM), 10 nM, 1 nM, 100 picomolar (pM), 10 pM, 1 pM, 100 femtomolar/L (fM), 10 fM, 1 fM or less growth factor. In some cases, a culture medium may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM growth factor. In some cases, a culture medium may comprise at most about 100 mM, 10 mM, 1 mM, 100 μM, 10 μM, 1 μM, 100 nM, 10 nM, 1 nM, 100 pM, 10 pM, 1 pM, 100 fM, 10 fM, 1 fM or less exogenous growth factor. In some cases, a culture medium may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM exogenous growth factor. In some cases, a culture medium may comprise at most about 100 mM, 10 mM, 1 mM, 100 μM, 10 μM, 1 μM, 100 nM, 10 nM, 1 nM, 100 pM, 10 pM, 1 pM, 100 fM, 10 fM, 1 fM or less endogenous growth factor. In some cases, a culture medium may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM endogenous growth factor. In some cases, a culture medium may comprise at most about 100 millimoles (mmoles), 10 mmoles, 1 mmole, 100 micromoles (μmoles), 10 μmoles, 1 μmole, 100 nanomoles (nmoles), 10 nmoles, 1 nmole, 100 picomoles (pmoles), 10 pmoles, 1 pmole, 100 femtomoles/L (fmoles), 10 fmoles, 1 fmole or less growth factor. In some cases, a culture medium may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole growth factor. In some cases, a culture medium may comprise at most about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, 1 fmole or less exogenous growth factor. In some cases, a culture medium may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole exogenous growth factor. In some cases, a culture medium may comprise at most about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, 1 fmole or less endogenous growth factor. In some cases, a culture medium may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole endogenous growth factor.

In some cases, a culture medium may comprise a limited amount of polypeptide encoded by an engineered nucleic acid (such as those described elsewhere in this disclosure). In some cases, a culture medium may not comprise a polypeptide encoded by an engineered nucleic acid. For example, in some cases, a culture medium may comprise at most about 100 milligrams per liter (mg/L), 10 mg/L, 1 mg/L, 100 micrograms per liter (μg/L), 10 μg/L, 1 μg/L, 100 nanograms per liter (ng/L), 10 ng/L, 1 ng/L, 100 picograms per liter (pg/L), 10 pg/L, 1 pg/L, 100 femtograms/L (fg/L), 10 fg/L, 1 fg/L or less polypeptide encoded by an engineered nucleic acid. In some cases, a culture medium may comprise at least about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L polypeptide encoded by an engineered nucleic acid. In some cases, a culture medium may comprise at most about 100 millimolar (mM), 10 mM, 1 mM, 100 micromolar (μM), 10 μM, 1 μM, 100 nanomolar (nM), 10 nM, 1 nM, 100 picomolar (pM), 10 pM, 1 pM, 100 femtomolar/L (fM), 10 fM, 1 fM or less polypeptide encoded by an engineered nucleic acid. In some cases, a culture medium may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM polypeptide encoded by an engineered nucleic acid. In some cases, a culture medium may comprise at most about 100 millimoles (mmoles), 10 mmoles, 1 mmole, 100 micromoles (umoles), 10 μmoles, 1 μmole, 100 nanomoles (nmoles), 10 nmoles, 1 nmole, 100 picomoles (pmoles), 10 pmoles, 1 pmole, 100 femtomoles/L (fmoles), 10 fmoles, 1 fmole or less polypeptide encoded by an engineered nucleic acid. In some cases, a culture medium may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole polypeptide encoded by an engineered nucleic acid.

In some instances, a culture medium without a polypeptide (such as those described elsewhere in this disclosure) or growth-factor, or comprising a limited amount of polypeptide or growth-factor, may comprise a culture medium prior to contacting a cell. In some instances, a culture medium without a polypeptide (such as those described elsewhere in this disclosure) or growth-factor, or comprising a limited amount of polypeptide or growth-factor, may comprise a culture medium that has not contacted a cell. In some cases, a culture medium comprising a cell or a growth of a cell may comprise a conditioned medium. In some cases, a conditioned medium may comprise a molecule derived from a cell. The molecule may be an endogenous or exogenous molecule of the cell. In some cases, the molecule derived from a cell may comprise a polypeptide. The polypeptide may be an endogenous or exogenous polypeptide of the cell. For example, an exogenous polypeptide of the cell may comprise a polypeptide that is encoded by an engineered nucleic acid. In other cases, an exogenous molecule of the cell may comprise a molecule that is synthesized or facilitated by a polypeptide that is encoded by an engineered nucleic acid. In some cases, an exogenous molecule or polypeptide of the cell may be a molecule or polypeptide derived from a cell that has been engineered, wherein the same molecule or polypeptide may not be derived from an otherwise comparable control cell that has not been engineered; in other cases, a cell that has not been engineered may generate at least about 10%, 20%, 30%, 40%, 50%, 60%, 70%, 80%, 90%, or 99% fewer of the same molecule or polypeptide described thereof, relative to a cell that has been engineered.

The polypeptide or exogenous polypeptide present within the culture medium may comprise any polypeptides encoded by the engineered nucleic acid described elsewhere in this disclosure.

In some cases, the polypeptide or exogenous polypeptide present within the culture medium in a limited amount or not present within the culture medium may bind a growth-factor receptor of a cell. The binding may activate the growth-factor receptor. The binding may inhibit the growth-factor receptor. In some cases, the polypeptide or exogenous polypeptide present within the culture medium in a limited amount or not present within the culture medium may activate a cell proliferation regulator of a cell. In some cases, the polypeptide or exogenous polypeptide present within the culture medium in a limited amount or not present within the culture medium may inhibit a cell proliferation regulator of a cell. In such cases, the binding, activation, or inhibition of the growth-factor receptor or cell proliferation regulator may increase, stimulate, or activate cell proliferation, cell migration, cell clustering, cell differentiation, or a combination thereof of a cell. The binding, activation, or inhibition of the growth-factor receptor or cell proliferation regulator may increase, stimulate, or activate a cell proliferation. The binding, activation, or inhibition of the growth-factor receptor or cell proliferation regulator may increase, stimulate, or activate a cell division. The binding, activation, or inhibition of the growth-factor receptor or cell proliferation regulator may increase, stimulate, or activate a cell growth. In other cases, a cell may comprise or be contacted with an engineered nucleic acid. The engineered nucleic acid may encode a variant of a cell proliferation regulator. While the cell proliferation regulator may be bound by the polypeptide or exogenous polypeptide present within the culture medium in a limited amount or not present within the culture medium to activate the proliferation of a cell, the variant of the cell proliferation regulator may activate the proliferation of a cell without such polypeptides. Such variants may comprise any variants described elsewhere in this disclosure.

A culture medium may have a volume of at least about 1 nanoliter (nL), 10 nL, 100 nL, 1 microliter (μL), 10 μL, 100 μL, 1 milliliter (mL), 10 mL, 100 mL, 1 liter (L), 10 L, 1×10\hat{ }2 L, 1×10\hat{ }3 L, 1×10\hat{ }4 L or more. A culture medium may have a volume of at most about 1 nL, 10 nL, 100 nL, 1 μL, 10 μL, 100 μL, 1 mL, 10 mL, 100 mL, 1 L, 10 L, 1×10\hat{ }2 L, 1×10\hat{ }3 L, or 1×10\hat{ }4 L.

A culture medium may have a pH of at least about 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, or 14. A culture medium may have a pH of at most about 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, or 14.

A culture medium may have an osmolarity of at least about 1 pico-osmoles per liter (pOsm/L), 10 pOsm/L, 100 pOsm/L, 1 nano-osmoles per liter (nOsm/L), 10 nOsm/L, 100 nOsm/L, 1 micro-osmoles per liter (μOsm/L), 10 μOsm/L, 100 μOsm/L, 1 milli-osmoles per liter (mOsm/L), 10 mOsm/L, 100 mOsm/L or more at 25° C., or an equivalent thereof at other temperatures. A culture medium may have an osmolarity of at most about 1 pOsm/L, 10 pOsm/L, 100 pOsm/L, 1 nOsm/L, 10 nOsm/L, 100 nOsm/L, 1μ Osm/L, 10μ Osm/L, 100μ Osm/L, 1 mOsm/L, 10 mOsm/L, or 100 mOsm/L at 25° C., or an equivalent thereof at other temperatures.

A culture medium may have an osmolarity of at least about 1 pico-osmoles per kilogram (pOsm/kg), 10 pOsm/kg, 100 pOsm/kg, 1 nano-osmoles per kilogram (nOsm/kg), 10 nOsm/kg, 100 nOsm/kg, 1 micro-osmoles per kilogram (μOsm/kg), 10 Osm/kg, 100 μOsm/kg, 1 milli-osmoles per kilogram (mOsm/kg), 10 mOsm/kg, 100 mOsm/kg or more at 25° C., or an equivalent thereof at other temperatures. A culture medium may have an osmolarity of at most about 1 pOsm/kg, 10 pOsm/kg, 100 pOsm/kg, 1 nOsm/kg, 10 nOsm/kg, 100 nOsm/kg, 1 μOsm/kg, 10 μOsm/kg, 100 μOsm/kg, 1 mOsm/kg, 10 mOsm/kg, or 100 mOsm/kg at 25° C., or an equivalent thereof at other temperatures.

A culture medium may have a temperature of at least about-200° C., −80° C., −70° C., −60° C., −50° C., −40° C., −30° C., −20° C., −10° C., −4° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or more. A culture medium may have a temperature of at most about −200° C., −80° C., −70° C., −60° C., −50° C., −40° C., −30° C., −20° C., −10° C., −4° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., or 37° C.

A culture medium may have a gaseous phase, a liquid phase, a solid phase, or a combination thereof. In some instances, a culture medium may comprise a serum. In some instances, a growth-factor within a culture medium may be derived from the serum. The serum may be derived from a primate. The serum may also be derived from a non-primate. The primate may be a non-human primate. In some cases, the serum may also be derived from a human. The serum may also be derived from an equine or a bovine. In some instances, a culture medium may comprise a serum-free medium. In some cases, a culture medium may comprise at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% serum, by dry weight of the serum per volume of the culture medium.

In some cases, the culture medium may comprise inorganic salts, amino acids, vitamins, additional chemical compounds, or a combination thereof. The inorganic salts of the culture medium may comprise ammonium metavanadate, ammonium molybdate-4H2O, calcium chloride, cupric sulfate-5H2O, ferric nitrate-9H2O, ferrous sulfate-7H2O, magnesium chloride-6H2O, magnesium sulfate (anhydrous), manganese sulfate, nickel chloride, potassium chloride, sodium bicarbonate, sodium chloride, sodium metasilicate-9H2O, sodium phosphate dibasic (anhydrous), sodium phosphate monobasic (anhydrous), sodium selenite, stannous chloride-2H2O, zinc sulfate-7H2O, or a combination thereof. The amino acids of the culture medium may comprise alanine, arginine, asparagine, aspartic acid, cysteine, cystine, glycine, glutamic acid, glutamine, histidine, isoleucine, leucine, lysine, methionine, phenylalanine, proline, serine, threonine, tryptophan, tyrosine, valine, or a combination thereof. The amino acids of the culture medium may be an L isomer. The amino acids of the culture medium may be a D isomer. The vitamins of the culture medium may comprise choline chloride, d-biotin, d-pantothenic acid (hemicalcium), folic acid, myo-inositol, niacinamide, pyridoxal-HCl, pyridoxine-HCl, riboflavin, thiamine-HCl, vitamin B12, or a combination thereof. The additional chemical compounds of the culture medium may comprise choline chloride, calcium chloride·2H2O, D-glucose, DL-thioctic acid, HEPES, hypoxanthine, linoleic acid, L-lysine·HCl, magnesium chloride-6H2O, magnesium sulfate, phenol red, putrescine·HCl, pyruvic acid, sodium bicarbonate, thymidine, or a combination thereof.

### Conditioned Medium

Provided herein are conditioned medium as described herein. A conditioned medium may comprise a culture medium that has been used to culture or grow a cell as described herein. For example, the cell may comprise an engineered nucleic acid as described herein (e.g., an RNA or DNA).

A conditioned medium may comprise a molecule synthesized by a cell as described herein. The molecule may comprise solvent(s), molecule(s), compound(s), metabolite(s), nutrient(s), and/or chemical(s) that can be used to culture or grow a cell. The molecule may comprise a polypeptide or peptide. The polypeptide or peptide may be encoded by an engineered nucleic acid of the cell. In some cases, the polypeptide or peptide may not be encoded by the engineered nucleic acid. In some cases, molecule may not be a polypeptide or peptide. For example, the engineered nucleic acid may facilitate the generation of the molecule by altering the cellular process of the cell.

In some cases, a conditioned medium (that has been used to culture a first cell) may be used to culture or grow a second cell. The first cell and second cell may be a same type of cell. The first cell and second cell may be different types of cell. The first cell may comprise a first engineered nucleic acid (such as any engineered nucleic acid as described herein) as described herein. The second cell may comprise a second engineered nucleic acid (such as any engineered nucleic acid as described herein). The first and second engineered nucleic acids may be a same engineered nucleic acid. The first and second engineered nucleic acids may be a different engineered nucleic acid. The second cell may not comprise any engineered nucleic acids. In some case, the second cell may be genetically modified. In other cases, the second cell may not be genetically modified.

Using a conditioned medium to culture a cell may have a beneficial advantage of minimizing a solvent(s), molecule(s), compound(s), metabolite(s), nutrient(s), and/or chemical(s) that can facilitate the culturing of the cell. In some cases, the conditioned medium that is generated by culturing or growing a cell may comprise a solvent(s), molecule(s), compound(s), metabolite(s), nutrient(s), and/or chemical(s) generated by the cell, and the conditioned medium may thus be used to culture a second cell (1) without adding the solvent(s), molecule(s), compound(s), metabolite(s), nutrient(s), and/or chemical(s); or (2) adding a reduced amount of the solvent(s), molecule(s), compound(s), metabolite(s), nutrient(s), and/or chemical(s), relative to a culture medium that has not been used to culture the cell.

For example, a conditioned medium (generated by a culture of a cell) may have at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, at least about 1000000-fold or more, by weight or concentration, of a molecule synthesized by the cell, relative to that of a culture medium that has not been used to culture or grow the cell. A conditioned medium (generated by a culture of a cell) may have at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold, by weight or concentration, of a molecule synthesized by the cell, relative to that of a culture medium that has not been used to culture or grow the cell

In some cases, the conditioned medium may comprise the molecule synthesized by the cell at a concentration of at least about 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M or more. In some cases, the conditioned medium may comprise the molecule synthesized by the cell at a concentration of at most about 1 pM, 10 pM, 100 pM, 1 nM, 10 nM, 100 nM, 1 μM, 10 μM, 100 μM, 1 mM, 10 mM, 100 mM, 1 M, 10 M, or 100 M.

In some cases, the conditioned medium may comprise the polypeptide or peptide encoded by the engineered nucleic acid at a concentration of at least about 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M or more. In some cases, the conditioned medium may comprise the polypeptide or peptide encoded by the engineered nucleic acid at a concentration of at most about 1 pM, 10 pM, 100 pM, 1 nM, 10 nM, 100 nM, 1 μM, 10 μM, 100 μM, 1 mM, 10 mM, 100 mM, 1 M, 10 M, or 100 M.

In some cases, the conditioned medium may comprise at least about 1 picoliter (pg), 10 μg, 100 pg, 1 nanoliter (ng), 10 ng, 100 ng, 1 microliter (μg), 10 μg, 100 μg, 1 milliliter (mg), 10 mg, 100 mg, 1 gram (g), 10 g, 100 g, 1 kilogram (kg), 10 kg or more of the polypeptide or peptide encoded by the engineered nucleic acid. In some cases, the conditioned medium may comprise at most about 1 picoliter (pg), 10 μg, 100 pg, 1 nanoliter (ng), 10 ng, 100 ng, 1 microliter (μg), 10 μg, 100 μg, 1 milliliter (mg), 10 mg, or 100 mg, 1 gram (g), 10 g, 100 g, 1 kilogram (kg), or 10 kg of the polypeptide or peptide encoded by the engineered nucleic acid.

In some cases, the conditioned medium may comprise at least about 1 picoliter (pg), 10 μg, 100 pg, 1 nanoliter (ng), 10 ng, 100 ng, 1 microliter (μg), 10 μg, 100 μg, 1 milliliter (mg), 10 mg, 100 mg, 1 gram (g), 10 g, 100 g, 1 kilogram (kg), 10 kg or more of the molecule synthesized by the cell. In some cases, the conditioned medium may comprise at most about 1 picoliter (pg), 10 μg, 100 pg, 1 nanoliter (ng), 10 ng, 100 ng, 1 microliter (μg), 10 μg, 100 μg, 1 milliliter (mg), 10 mg, or 100 mg, 1 gram (g), 10 g, 100 g, 1 kilogram (kg), or 10 kg of the molecule synthesized by the cell.

In some cases, the conditioned medium may have a volume of at least about 1 picoliter (pL), 10 μL, 100 pL, 1 nanoliter (nL), 10 nL, 100 nL, 1 microliter (μL), 10 μL, 100 μL, 1 milliliter (mL), 10 mL, 100 mL, 1 liter (L), 10 L, 100 L or more. In some cases, the conditioned medium may have a volume of at most about 1 picoliter (pL), 10 μL, 100 pL, 1 nanoliter (nL), 10 nL, 100 nL, 1 microliter (μL), 10 μL, 100 μL, 1 milliliter (mL), 10 mL, 100 mL, 1 liter (L), 10 L, or 100 L.

In some cases, generating a conditioned medium may comprise culturing or growing the cell for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month or more. In some cases, generating a conditioned medium may comprise culturing or growing the cell for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 1 day, at most about 2 days, at most about 3 days, at most about 4 days, at most about 5 days, at most about 6 days, at most about 1 week, at most about 2 weeks, at most about 3 weeks, or at most about 1 month.

When using the conditioned medium to culture a second cell, the conditioned medium may be mixed with a culture medium that has not been used to culture a cell. In some cases, the conditioned medium may be mixed with a different conditioned medium that has been used to culture a cell different from the second cell. In some cases, when using the conditioned medium to culture a cell, the conditioned medium may be mixed with solvents, molecules, compounds, metabolites, nutrients, and/or chemicals for supporting the growth of a cell in vitro or ex vivo. For example, because growing a first cell with the culture medium may deplete certain solvents, molecules, compounds, metabolites, nutrients, and/or chemicals from the culture medium, the conditioned medium may thus be replenished with the depleted solvents, molecules, compounds, metabolites, nutrients, and/or chemicals when used for culturing the second cell.

In some cases, the conditioned medium may not be filtered when used to culture a cell. In some cases, the conditioned medium may be dialyzed when used to culture a cell. In some cases, the conditioned medium may not be dialyzed when used to culture a cell. In some cases, the conditioned medium may be filtered when used to culture a cell. In some cases, the conditioned medium may not be sterilized when used to culture a cell. In some cases, the conditioned medium may be sterilized when used to culture a cell. Filtering, dialyzing, or sterilizing may remove any molecules or contaminations that negatively impacts the growth of the cell. Sterilizing may comprise heating (or autoclaving) the conditioned medium. In some case, the conditioned medium (that has been used to culture or grow a first cell) for growing a second cell may comprise the first cell. In some case, the conditioned medium (that has been used to culture or grow a first cell) for growing a second cell may be cell-free (i.e., not comprising the first cell). In some cases, the conditioned medium may comprise the first cell that is inviable. For example, sterilizing the conditioned medium may generate the inviable cell in the conditioned medium.

### Contacting Cells

In some cases, a method may comprise contacting a cell with an engineered nucleic acid. In some cases, the contacting may comprise transfecting the cell with the engineered nucleic acid. In some cases, the transfecting may comprise the cell taking up the engineered nucleic acid. The method may comprise contacting the cell prior to the cell being present in the culture medium. The method may comprise contacting the cell during the cell being present in the culture medium. The method may comprise contacting the cell subsequent to the cell being present in the culture medium.

In some instances, the engineered nucleic acid contacting a cell may not be encapsulated by a vesicle. The engineered nucleic acid contacting a cell may not be encapsulated by a micro-vesicle. The engineered nucleic acid contacting a cell may not be encapsulated by an extracellular vesicle. The extracellular vesicle may comprise a lipid-based vesicle derived from a cell other than the cell being contacted with the engineered nucleic acid. In some cases, the engineered nucleic acid contacting a cell may not be encapsulated by an exogenous vesicle, micro-vesicle, or extracellular vesicle derived from the cell. The engineered nucleic acid may not be encapsulated by an exosome. The engineered nucleic acid may not be encapsulated by an apoptotic bodies. In some instances, the engineered nucleic acid contacting a cell may not be encapsulated by any vesicles described thereof prior to growing the cell comprising an engineered nucleic acid. In some cases, the engineered nucleic acid contacting a cell may be encapsulated by a vesicle, micro-vesicle, extracellular vesicle, exosome, apoptotic body, or a combination thereof.

In some cases, the contacting may comprise contacting the cell with the engineered nucleic acid, a saccharide, or a polymeric material in a condition sufficient (i.e., a sufficient condition) for the cell to uptake the engineered nucleic acid, the saccharide, the polymeric material, or the combination thereof. In some cases, the saccharide, the engineered nucleic acid, the polymeric material may associate with each other to form a polyplex. The polyplex may be different from the vesicles that can encapsulate the engineered nucleic acid as described elsewhere in this disclosure. The polyplex, or the saccharide or polymeric material that associates with the engineered nucleic prior to or during the contacting may be a derivative of an engineered nucleic acid.

In some cases, a sufficient condition may comprise contacting a cell with an engineered nucleic acid or a derivative thereof for at least about 0.1, 02, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 hours or more. In some cases, a sufficient condition may comprise contacting a cell with an engineered nucleic acid for at most about 0.1, 02, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, or 24 hours. In some cases, a sufficient condition may comprise contacting a cell with an engineered nucleic acid or a derivative thereof for at least about 1, 2, 3, 4, 5 days or more. In some cases, a sufficient condition may comprise contacting a cell with an engineered nucleic acid for at most about 1, 2, 3, 4, or 5 days. In some cases, a sufficient condition may comprise contacting a cell with an engineered nucleic acid, a saccharide, or a combination thereof for at least about 1, 2, 3, 4, 5 days or more.

In some cases, a sufficient condition may comprise contacting a cell with an engineered nucleic acid or a derivative thereof at a temperature of at least about-200° C., −80° C., −70° C., −60° C., −50° C., −40° C., −30° C., −20° C., −10° C., −4° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C. or more. A sufficient condition may comprise contacting a cell with an engineered nucleic acid or a derivative thereof at a temperature of at most about-200° C., −80° C., −70° C., −60° C., −50° C., −40° C., −30° C., −20° C., −10° C., −4° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., or 43° C.

In some cases, the contacting may be carried out in a two-dimensional (2D) or three-dimensional (3D) contacting medium. In other cases, the cell being contacted may be agitated. In some cases, the cell being contact may not be agitated.

In some instances, the contacting may comprise contacting a cell with at least 1 engineered nucleic acid. In other case, the contacting may comprise contacting a cell with more than 1 engineered nucleic acid. For example, the contacting may comprise contacting a cell with at least about 2, 3, 4, 5, 6, 7, 8, 9, 10, 11, 12, 13, 14, 15, 16, 17, 18, 19, 20 or more engineered nucleic acids. The contacting may comprise contacting a cell with at least about 2, 3, 4, 5, 6, 7, 8, 9, 10, 11, 12, 13, 14, 15, 16, 17, 18, 19, or 20 engineered nucleic acids. In some cases, the multiple engineered nucleic acids may comprise a same coding sequence, a different coding sequence, or a combination thereof. In some cases, the multiple engineered nucleic acids may comprise a same engineered nucleic acid (i.e., the multiple engineered nucleic acids may comprise copies of a same engineered nucleic acid). In other cases, the multiple engineered nucleic acids may comprise different engineered nucleic acids.

In some cases, the contacting may be carried out at an atmospheric pressure of less than 25% oxygen, such as less than 24%, 23%, 22%, 21%, 20%, 19%, 18%, 17%, 16%, 15%, 10%, 5%, or 0% oxygen.

### Growing or Culturing Cells

In some instances, the method may comprise growing (or culturing) a cell within a culture medium. Growing the cell may comprise increasing a cell number, a cell mass, or a combination thereof within a culture medium. In some cases, growing the cell may comprise proliferating a cell within a culture medium. In some cases, growing the cell may comprise the cell undergoing a cell division, a cell growth, or a combination thereof within a culture medium.

In some instances, growing the cell may comprise increasing a cell number to at least about 2, 3, 4, 5, 6, 7, 8, 9, 10, 20, 30, 40, 50, 60, 70, 80, 90, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, 10\hat{ }15 or more. In some instances, growing the cell may comprise increasing a cell number to at most about 2, 3, 4, 5, 6, 7, 8, 9, 10, 20, 30, 40, 50, 60, 70, 80, 90, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, or 10\hat{ }15. In some instances, growing the cell may comprise increasing a cell number to at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, 1000000-fold, or more, higher than the cell number prior to growing the cell. In some instances, growing the cell may comprise increasing a cell number to at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than the cell number prior to growing the cell. In some instances, growing the cell may comprise increasing a cell mass to at least about 1 nanogram (ng), 10 ng, 100 ng, 1 microgram (μg), 10 μg, 100 μg, 1 milligram (mg), 10 mg, 100 mg, 1 gram (g), 10 g, 100 g, 1 kilogram (kg), 10 kg or more, by dry weight of the cell. In some instances, growing the cell may comprise increasing a cell mass to at most about 1 ng, 10 ng, 100 ng, 1 μg, 10 μg, 100 μg, 1 mg, 10 mg, 100 mg, 1 g, 10 g, 100 g, 1 kg, or 10 kg, by dry weight of the cell. In some instances, growing the cell may comprise increasing a cell mass to at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, 1000000-fold, or more, higher relative to the cell mass prior to growing the cell. In some instances, growing the cell may comprise increasing a cell mass to at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than the cell mass prior to growing the cell. In some cases, growing the cell may comprise increasing any combination of cell masses and cell numbers described thereof.

In some instances, a cell comprising an engineered nucleic acid may exhibit a higher proliferation rate relative to a cell that does not comprise the engineered nucleic acid. In some cases, an engineered cell may exhibit a higher proliferation rate relative to a cell not engineered. In some cases, a proliferation rate may comprise the time taken for a cell to complete a cell division within a culture medium, the time taken for a cell to exhibit a pre-determined increase of cell mass within a culture medium, the number of cell divisions that a cell can undergo within a culture medium, the amount of cell mass that a cell can attain within a culture medium, or a combination thereof. In some cases, a proliferation rate may comprise the time taken for a cell to complete a cell division within a culture medium. In some cases, a proliferation rate may comprise the time taken for a cell to exhibit a pre-determined increase of cell mass within a culture medium. In some cases, a proliferation rate may comprise the number of cell divisions that a cell can undergo within a culture medium.

In some instances, a cell comprising an engineered nucleic acid may exhibit a proliferation rate at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that does not comprise the engineered nucleic acid. In some cases, a cell comprising an engineered nucleic acid may exhibit a proliferation rate at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that does not comprise the engineered nucleic acid. In some instances, an engineered cell may exhibit a proliferation rate at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that is not engineered. In some cases, an engineered cell may exhibit a proliferation rate at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that is not engineered.

In some instances, a cell comprising an engineered nucleic acid may increase the cell number to at least about 2, 3, 4, 5, 6, 7, 8, 9, 10, 20, 30, 40, 50, 60, 70, 80, 90, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, 10\hat{ }15 or more. In some instances, a cell comprising an engineered nucleic acid may increase the cell number to at most about 2, 3, 4, 5, 6, 7, 8, 9, 10, 20, 30, 40, 50, 60, 70, 80, 90, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, or 10\hat{ }15. In some instances, a cell comprising an engineered nucleic acid may increase the cell mass to at least about 1 ng, 10 ng, 100 ng, 1 μg, 10 μg, 100 μg, 1 mg, 10 mg, 100 mg, 1 g, 10 g, 100 g, 1 kg, or 10 kg, by dry weight of the cell. In some instances, a cell comprising an engineered nucleic acid may increase the cell mass to at most about 1 ng, 10 ng, 100 ng, 1 μg, 10 μg, 100 μg, 1 mg, 10 mg, 100 mg, 1 g, 10 g, 100 g, 1 kg, or 10 kg, by dry weight of the cell.

In some instances, a cell comprising an engineered nucleic acid may exhibit a proliferation rate at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of the cell that does not comprise the engineered nucleic acid. In some cases, a cell comprising an engineered nucleic acid may exhibit a proliferation rate at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of the cell that does not comprise the engineered nucleic acid.

In some instances, a cell comprising an engineered nucleic acid may comprise a cell line. In some cases, a cell that is not a cell line may be converted to the cell line after being contacted with and taking up an engineered nucleic acid. A cell line may comprise a cell or a group of cells that proliferates, in vitro, ex vivo, or outside of an organism or a host, for a substantially larger extent than that of an otherwise comparable cell (e.g., the same cell that does not comprise the engineered nucleic acid) that is not a cell line. A cell line may be immortal. A cell line may not undergo cellular senescence. A cell line may also be highly proliferative. In some instances, a cell line may exhibit a proliferation rate at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that is not a cell line. In some cases, a cell line may exhibit a proliferation rate at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell that is not a cell line.

In some instances, growing a cell may generate a population of a cell. The population of the cell may comprise the cell, the progenies of the cell, or a combination thereof. In some cases, a progeny of a cell comprising an engineered nucleic acid may comprise the engineered nucleic acid. In some case, a progeny of a cell comprising an engineered nucleic acid may not comprise the engineered nucleic acid. In some instances, a cell, a progeny, or a population of cells that is derived from a cell comprising an engineered nucleic acid may exhibit a proliferation rate at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell, a progeny, or a population of cells not derived from the cell comprising the engineered nucleic acid. In some cases, a cell, a progeny, or a population of cells derived from a cell comprising an engineered nucleic acid may exhibit a proliferation rate at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher than a proliferation rate of a cell, a progeny, or a population of cells not derived from the cell that does not comprise the engineered nucleic acid.

In some instances, a population of progenies or cells derived from at least a cell comprising an engineered nucleic acid may comprise cells that comprise the engineered nucleic acid and cells that do not comprise the engineered nucleic acid. In some instances, the population of the cell may proliferate within a culture medium not comprising an exogenous growth factor or comprising a limited amount of the exogenous growth factor. In some cases, even if the exogenous growth factor is not provided or provided as a limited amount to the culture medium, at least a subset of the population of the cells may generate a polypeptide that is at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the exogenous growth-factor. The subset of the population of the cells may also generate a polypeptide that is at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the exogenous growth-factor. In other cases, the subset of the population of the cells may generate a polypeptide that can bind to a same growth-factor receptor that is bound by the exogenous growth factor; or activate and inhibit a same cell proliferation regulator that is activated and inhibited by the exogenous growth factor, respectively. In this case, the polypeptide and the exogenous growth-factor may share a sequence identity described thereof. In this case, the polypeptide and the exogenous growth-factor may not share any sequence identity.

In some instances, a population of cells derived from at least a cell comprising an engineered nucleic acid may comprise at least a subset of cells that generate a second polypeptide. The second polypeptide may comprise a growth-factor. The second polypeptide may comprise any polypeptides described elsewhere in this disclosure. In some cases, the second polypeptide may be secreted by one of the subset of cells. In some cases, the second polypeptide may be encapsulated by a vesicle derived from one of the subset of cells. The second polypeptide may be encapsulated by a micro-vesicle derived from one of the subset of cells. The second polypeptide may be encapsulated by an extracellular vesicle derived from one of the subset of cells. In some cases, The second polypeptide may be encapsulated by an exogenous vesicle, micro-vesicle, or extracellular vesicle derived from one of the subset of cells. The engineered nucleic acid may be encapsulated by an exosome derived from one of the subset of cells. The engineered nucleic acid may be encapsulated by an apoptotic bodies derived from one of the subset of cells. The second polypeptide encapsulated may contact or be taken up by the cells in the populations of cells. In some cases, the second polypeptide encapsulated may facilitate the proliferation of the cells that has contacted or taken up the second polypeptide. In some cases, the second polypeptide may be secreted by one of the subset of cells, wherein the second polypeptide is not encapsulated by any vesicles described thereof. In such cases, the second polypeptide secreted but not encapsulated may contact or be taken up by the cells in the populations of cells. In some cases, the second polypeptide secreted but not encapsulated may facilitate the proliferation of the cells that has contacted or taken up the second polypeptide.

In some instances, a population of cells derived from at least a cell comprising an engineered nucleic acid may comprise the cells that comprise the engineered nucleic acid and cells that do not comprise the engineered nucleic acid. The engineered nucleic acid may encode a growth-factor. The engineered nucleic acid may encode any polypeptides described elsewhere in this disclosure. The engineered nucleic acid may comprise RNA. In some cases, the engineered nucleic acid may be secreted by the cells that comprise the engineered nucleic acid. In some cases, the engineered nucleic acid may be encapsulated by a vesicle derived from the cells that comprise the engineered nucleic acid. The engineered nucleic acid may be encapsulated by a micro-vesicle derived from the cells that comprise the engineered nucleic acid. The engineered nucleic acid may be encapsulated by an extracellular vesicle derived from the cells that comprise the engineered nucleic acid. In some cases, The engineered nucleic acid may be encapsulated by an exogenous vesicle, micro-vesicle, or extracellular vesicle derived from the cells that comprise the engineered nucleic acid. The engineered nucleic acid may be encapsulated by an exosome derived from the cells that comprise the engineered nucleic acid. The engineered nucleic acid may be encapsulated by an apoptotic bodies derived from the cells that comprise the engineered nucleic acid. The engineered nucleic acid encapsulated may be taken up by the cells in the populations of cells. In some cases, a cell that does not comprise the engineered nucleic acid may take up the engineered nucleic acid, and the cell may generate the polypeptide encoded by the engineered nucleic acid. In some cases, the engineered nucleic acid may facilitate the proliferation of the cells that have taken up the engineered nucleic acid.

In some cases, the method may comprise growing a cell for at least about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, 6 months or more. In some cases, the method may comprise growing a cell for at most about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, or 6 months. In some cases, the method may comprise growing a cell for at least about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, 6 months or more after contacting cell with an engineered nucleic acid. In some cases, the method may comprise growing a cell for at most about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, or 6 months after contacting cell with an engineered nucleic acid.

In some cases, the method may comprise growing a cell at an atmospheric pressure of less than 25% oxygen, such as less than 24%, 23%, 22%, 21%, 20%, 19%, 18%, 17%, 16%, 15%, 10%, 5%, or 0% oxygen.

In some cases, the method may comprise growing a cell at a temperature that is at least about 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C. or more. In some cases, the method may comprise growing a cell at a temperature that is at most about 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C.

In some cases, the method may comprise growing the cell in a 2D or 3D environment. In some cases, the cell may be grown in the culture medium without a scaffold. In other cases, the cell may also be grown in the culture medium with a scaffold. In some cases, a cell may be viable. A cell may also be healthy. In some cases, a sufficient condition may comprise a population of viable cells before the contacting. The population of cells may comprise at least about 10%, 20%, 30%, 40%, 50%, 60%, 70%, 80%, 90%, 99% or more viable cells. The population of cells may also comprise from about 80% to about 90% viable cells. The population of cells may comprise at most about 10%, 20%, 30%, 40%, 50%, 60%, 70%, 80%, 90%, or 100% viable cells.

In some cases, the growing may allow a cell in a culture medium to attain to a desired density. A desired density, in some cases, may comprise at least about 1×10\hat{ }4, 2×10\hat{ }4, 5×10\hat{ }4, 1×10\hat{ }5, 2×10\hat{ }5, 5×10\hat{ }5, 1×10\hat{ }6, 2×10\hat{ }6, 5×10\hat{ }6, 1×10\hat{ }7, 2×10\hat{ }7, 5×10\hat{ }7, 1×10\hat{ }8, 2×10\hat{ }8, 5×10\hat{ }8, 1×10\hat{ }9, 2×10\hat{ }9, 5×10\hat{ }9, 1×10\hat{ }10, 2×10\hat{ }10, 5×10\hat{ }10, 1×10\hat{ }11, 2×10\hat{ }11, 5×10\hat{ }11, 1×10\hat{ }12, 2×10\hat{ }12, 5×10\hat{ }12, 1×10\hat{ }13, 2×10\hat{ }13, 5×10\hat{ }13, 1×10\hat{ }14, 2×10\hat{ }14, 5×10\hat{ }14, 1×10\hat{ }15, 2×10\hat{ }15, 5×10\hat{ }15 or more cells/ml of the culture medium. A desired density, in some cases, may comprise at most about 1×10\hat{ }4, 2×10\hat{ }4, 5×10\hat{ }4, 1×10\hat{ }5, 2×10\hat{ }5, 5×10\hat{ }5, 1×10\hat{ }6, 2×10\hat{ }6, 5×10\hat{ }6, 1×10\hat{ }7, 2×10\hat{ }7, 5×10\hat{ }7, 1×10\hat{ }8, 2×10\hat{ }8, 5×10\hat{ }8, 1×10\hat{ }9, 2×10\hat{ }9, 5×10\hat{ }9, 1×10\hat{ }10, 2×10\hat{ }10, 5×10\hat{ }10, 1×10\hat{ }11, 2×10\hat{ }11, 5×10\hat{ }11, 1×10\hat{ }12, 2×10\hat{ }12, 5×10\hat{ }12, 1×10\hat{ }13, 2×10\hat{ }13, 5×10\hat{ }13, 1×10\hat{ }14, 2×10\hat{ }14, 5×10\hat{ }14, 1×10\hat{ }15, 2×10\hat{ }15, or 5×10\hat{ }15 cells/ml of the culture medium.

### Expressing Polypeptides

In some instances, the method may comprise expressing a polypeptide by a cell, or a progeny thereof. In some cases, the polypeptide may be encoded by a nucleic acid of the cell. In other cases, the polypeptide may be encoded by an engineered nucleic acid. The polypeptide may also not be encoded by an engineered nucleic acid. For example, a first polypeptide may be expressed by a cell that comprises the engineered nucleic acid, wherein the engineered nucleic acid may encode a second polypeptide that facilitates the expression of the first polypeptide by the cell.

In some cases, the method may comprise expressing a polypeptide by a cell to a desired amount.

The desired amount of the polypeptide may comprise at least about 100 mg, 10 mg, 1 mg, 100 μg, 10 μg, 1 μg, 100 ng, 10 ng, 1 ng, 100 μg, 10 pg, 1 μg, 100 fg, 10 fg, or 1 fg of the polypeptide by dry weight. The desired amount of the polypeptide may comprise at most about 100 mg, 10 mg, 1 mg, 100 μg, 10 μg, 1 μg, 100 ng, 10 ng, 1 ng, 100 μg, 10 pg, 1 μg, 100 fg, 10 fg, or 1 fg of the polypeptide by dry weight. The desired amount of the polypeptide may comprise at least about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L of the polypeptide per solvent or volume of the container. The desired amount of the polypeptide may comprise at most about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L of the polypeptide per solvent or volume of the container. The desired amount of the polypeptide may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM of the polypeptide in a solvent. The desired amount of the polypeptide may comprise at most about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM of the polypeptide in a solvent. The desired amount of the polypeptide may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole of the polypeptide. In some cases, a culture medium may comprise at most about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, 1 fmole of the polypeptide.

In some cases, the expressing may comprise expressing a polypeptide by a cell for at least about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, 6 months or more. In some cases, the method may comprise expressing a polypeptide by a cell for at most about 1, 2, 3, 4, 5, 6, 7, 8, 9, 10, 11, 12, 13, 14, 15, 16, 17, 18, 19, 20, 21, 22, 23, 24 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 2, 3, 4, 5, or 6 months. During the time when the cell is expressing the polypeptide, the cell may be proliferating. In other cases, the cell may not be proliferating during the time when the cell is expressing the polypeptide.

In some cases, the method may comprise expressing the polypeptide by the cell at a temperature that is at least about 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C. or more. In some cases, the method may comprise expressing the polypeptide by the cell at a temperature that is at most about 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C.

### Harvesting

In some cases, the method may comprise harvesting a molecule synthesized by the cell. In some cases, the method may comprise harvesting a cell that has synthesized the molecule. In some cases, the method may comprise harvesting a polypeptide expressed by the cell. In some cases, the method may comprise harvesting a cell that has expressed the polypeptide. In some cases, the method may comprise harvesting a cell or cell population comprising the engineered nucleic acid. In some cases, the method may comprise harvesting a cell or cell population derived from a cell comprising the engineered nucleic acid. In some cases, the cell or cell population may comprise the engineered nucleic acid. In some cases, the cell or cell population may not comprise the engineered nucleic acid. The polypeptide harvested may comprise any polypeptide described herein. For example, the polypeptide may be encoded by the engineered nucleic acid. In some cases, the polypeptide may not be encoded by the engineered nucleic acid.

The molecule or polypeptide may be released by the cell into the culture medium or a solvent. In some cases, the molecule or polypeptide may be secreted by the cell. In some cases, the molecule or polypeptide may be released into the cultured medium or the solvent by subjecting the culture with the cell with a treatment. The treatment may comprise an enzymatic treatment, a digestion treatment, a heating treatment, a sonication treatment, a mechanic treatment, or a combination thereof.

In some instances, the harvesting may comprise extracting the polypeptide or derivative thereof from the cell, extracting the polypeptide from the culture medium, extracting a cell or progeny thereof from the culture medium, or a combination thereof. In other cases, the harvesting may comprise extracting the polypeptide from the culture medium. In some cases, the harvesting may comprise extracting the cell from the culture medium.

In some instances, an entity being extracted may be purified. As described thereof, the entity may comprise a cell, a polypeptide, or a combination thereof. In some case, the purified entity may comprise at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher concentration of the entity, relative to the concentration of the entity prior to being purified. In some case, the purified entity may comprise at most about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, 5-fold, 10-fold, 50-fold, 100-fold, 500-fold, 1000-fold, 10000-fold, 100000-fold, or 1000000-fold higher concentration of the entity, relative to the concentration of the entity prior to being purified.

In some case, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90 91%, 92%, 93%, 94%, 95%, 96%, 97%, 98%, 99%, or 100% of the molecules in the purified entity may comprise the molecules of the entity. In some case, at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the molecules in the purified entity may comprise the molecules of the entity. In other cases, at most about 0%, 1%, 2%, 3%, 4%, 5%, 6%, 7%, 8%, 9%, 10%, 15%, 20%, 25%, 30%, 35%, 40%, 45%, 50% of the molecules in the purified entity may comprise the molecules not of the entity.

In some cases, the harvesting may comprise obtaining a desired amount of the harvested entity.

The desired amount of the harvested entity may comprise at least about 100 mg, 10 mg, 1 mg, 100 μg, 10 μg, 1 μg, 100 ng, 10 ng, 1 ng, 100 μg, 10 pg, 1 μg, 100 fg, 10 fg, or 1 fg of the harvested entity by dry weight. The desired amount of the harvested entity may comprise at most about 100 mg, 10 mg, 1 mg, 100 μg, 10 μg, 1 μg, 100 ng, 10 ng, 1 ng, 100 μg, 10 pg, 1 μg, 100 fg, 10 fg, or 1 fg of the harvested entity by dry weight. The desired amount of the harvested entity may comprise at least about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L of the harvested entity per solvent or volume of the container. The desired amount of the harvested entity may comprise at most about 100 mg/L, 10 mg/L, 1 mg/L, 100 μg/L, 10 μg/L, 1 μg/L, 100 ng/L, 10 ng/L, 1 ng/L, 100 pg/L, 10 pg/L, 1 pg/L, 100 fg/L, 10 fg/L, or 1 fg/L of the harvested entity per solvent or volume of the container. The desired amount of the harvested entity may comprise at least about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM of the harvested entity in a solvent. The desired amount of the harvested entity may comprise at most about 100 mM, 10 mM, 1 mM, 100 μM, 10 μM, 1 μM, 100 nM, 10 nM, 1 nM, 100 pM, 10 pM, 1 pM, 100 fM, 10 fM, or 1 fM of the harvested entity in a solvent. The desired amount of the harvested entity may comprise at least about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, or 1 fmole of the harvested entity. In some cases, a culture medium may comprise at most about 100 mmoles, 10 mmoles, 1 mmole, 100 μmoles, 10 μmoles, 1 μmole, 100 nmoles, 10 nmoles, 1 nmole, 100 pmoles, 10 pmoles, 1 pmole, 100 fmoles, 10 fmoles, 1 fmole of the molecules of the harvested entity.

In other cases, the harvesting may comprise harvesting a cell that has been converted into a therapeutic product. The harvesting may comprise harvesting a cell that has been converted into a meat product.

Converting Cells into Meat Products

In some instances, a method may comprise converting the cell or the progeny thereof into a meat product. The cell may comprise a cell comprising an engineered nucleic acid or a derivative thereof.

The converting may comprise converting a cell from a cell type into another cell type. In some cases, the converting may comprise converting an undifferentiated cell into a differentiated cell. In some cases, the converting may comprise converting a differentiated cell into an undifferentiated cell. In some cases, the converting may comprise converting a cell of a first differentiated type into a cell of a second differentiated type, wherein the first and second differentiated types are different. In some instances, the method may further comprise growing a cell that has been converted.

A meat product may comprise any cells or combinations of cells described elsewhere in this disclosure. In some instances, a meat product may comprise a muscle cell, a fat cell, a connective tissue cell, a vasculature cell, a neuron, a bone cell, a skin cell, or a combination thereof. A meat product may comprise a muscle cell. A meat product may comprise a fat cell. A meat product may comprise a connective tissue cell. A meat product may comprise a vasculature cell. A meat product may comprise a neuron. A meat product may comprise a bone cell. A meat product may comprise a skin cell.

A meat product may comprise a tissue. A tissue may comprise a collection of cells. The cells of a tissue may be from one cell type. The cells of a tissue may be from more than one cell type. In some cases, a tissue or group of cells may form an organ. In some cases, the group of tissues or cells of an organ may be found in an animal. In some cases, the tissues or cells from an organ may collectively perform a physiological or cellular function. In some cases, the tissues or cells from an organ may share structural and/or functional characteristics. In some cases, an organ may comprise a bladder, a blood vessel, a bone, a brain, a bronchi, a cartilage, a diaphragm, a fallopian tube, a gill, a hair, a heart, a hypothalamus, an intestine, a kidney, a larynx, a ligament, a liver, a lung, a lymph node, a muscle, a nail, a nerve, an ovary, a pancreas, a parathyroid, a penis, a pharynx, a pineal body, a pituitary gland, a prostate, a scale, a skin, a spinal cord, a spleen, a stomach, a tendon, a testis, a thymus, a thyroid, a tonsil, a tooth, a trachea, a ureters, a urethra, a vagina, a vas deferens, a vulva, or a combination thereof. In some cases, a tissue or group of cells may form an organoid. In some cases, the organoid may be formed in an in vitro or ex vivo culture. In some cases, the organoid may be 3D. In some cases, the tissues or cells from an organoid may collectively perform a physiological or cellular function. In some cases, the tissues or cells from an organoid may share structural and/or functional characteristics. In some cases, a model may comprise a cell or a collection of cells. In some cases, a model comprising a cell or a collection of cells may be a model of the organ, the organoid, the tissue, or a combination thereof. In some cases, a model comprising a cell or a collection of cells may be a model of the organ. In some cases, a model comprising a cell or a collection of cells may be a model of the organoid. In some cases, a model comprising a cell or a collection of cells may be a model of the tissue. In some cases, a model may have at least one functional and/or structural characteristic of the cell, the collection of cells, the tissue, the organ, the organoid, or a combination thereof. In some cases, a model may comprise organic or inorganic materials. In some cases, a model may comprise a cell, tissue, organ, or organoid; or a component derived thereof. In some cases, a model may be a biomimetic model.

A meat product may also comprise a group of cells. A meat product may also comprise a group of cells having a population of cell types of a tissue from an animal. A meat product may be meat when it is consumed by any species, and it causes no harm. Such harm may comprise poisoning, contamination, or infection. The edibility of a meat may depend on the types or properties of the meat. For example, the meats may be processed or cooked to be meat. In some cases, meats may be baked, steamed, poached, boiled, grilled, dried, smoked, fried, heated, pickled, fermented, aged, or any combination thereof. Some meats, such as those from fish, may be meat without cooking. The edibility of a meat may depend on the level of toxins and contaminating organisms. In some cases, the edibility of a meat may also depend on the appetite of a person consuming the meat products.

In some cases, the expressing polypeptides, the harvesting, the contacting or a combination thereof may be carried out at a partial pressure of less than 25% oxygen, such as less than 24%, 23%, 22%, 21%, 20%, 19%, 18%, 17%, 16%, 15%, 10%, 5%, or 0% oxygen.

### Cells

In some instances, a cell described in this disclosure may comprise a fat cell, a blood vessel cell, a cardiac cell, a chondrocyte, an endothelial cell, an epithelial cell, a hematopoietic cell, a hepatocyte, a muscle cell, a neuron, or an osteogenic cell. In some cases, a cell may comprise a fat cell. In some cases, a cell may comprise a blood vessel cell. In some cases, a cell may comprise a cardiac cell. In some cases, a cell may comprise a chondrocyte. In some cases, a cell may comprise an endothelial cell. In some cases, a cell may comprise an epithelial cell. In some cases, a cell may comprise a hematopoietic cell. In some cases, a cell may comprise a hepatocyte. In some cases, a cell may comprise a muscle cell. In some cases, a cell may comprise a neuron. In some cases, a cell may comprise an osteogenic cell. In some instances, a cell may comprise at least 2, 3, 4, 5, 6, 7, 8, 9, 10, or 11 of a fat cell, a blood vessel cell, a cardiac cell, a chondrocyte, an endothelial cell, an epithelial cell, a hematopoietic cell, a hepatocyte, a muscle cell, a neuron, and an osteogenic cell. In some cases, a cementogenic cell comprises a cell that can form or be a part of a tooth. In some cases, an endothelial cell comprises a cell that can form or be a part of the endothelium. In some cases, an epithelial cell comprises a cell that can form or be a part of the epithelium. In some cases, a fat cell may comprise a cell that can form or be a part of the adipose tissues. In some cases, a hematopoietic cell may comprise a cell that can form or be a part of the blood or hematopoietic tissues. In some cases, a hepatocyte may comprise a cell that can form or be a part of the hepatic or liver tissue. In some cases, a muscle cell may comprise a cell that can form or be a part of the muscle tissues. In some cases, a neuron may comprise a cell that can form or be a part of the nervous systems. In some cases, a renal cell may comprise a cell that can form or be a part of the kidney tissues. In some cases, a retinal cell may comprise a cell that can form or be a part of the ocular or eye tissues. In some cases, an osteogenic cell may comprise a cell that can form or be a part of the bone tissues. In some cases, a blood vessel cell or an angiogenic cell may comprise a cell that can form or be a part of the blood vessel or vascular tissue. In some cases, a cardiac cell may comprise a cell that can form or be a part of the heart or cardiac tissue. In some cases, a chondrocyte cell may comprise a cell that can form or be a part of the cartilage tissue.

In some cases, a fat cell may be an adipocyte. In some cases, a fat cell may contain various sizes of fat droplets or granules. In some cases, a fat cell may comprise a white adipose cell or a brown adipose cell. In some cases, a white adipose cell may contain large fat droplets or granules and a small amount of cytoplasm. In some cases, a brown adipose cell may contain a large amount of cytoplasm and numerous mitochondria. In some cases, an adipocyte may be a cell primarily composed of adipose tissue, specialized in synthesizing and storing energy as fat. Adipocytes may be derived from induced pluripotent stem cells and/or mesenchymal stem cells through adipogenesis. Adipocytes may be white adipocytes, which store energy as a single large lipid droplet and have important endocrine functions, and brown adipocytes which store energy in multiple small lipid droplets but specifically for use as fuel to generate body heat.

In some cases, a muscle cell may develop sarcoplasm, sarcoplasmic reticulum, sarcosome, or sarcolemma that are specialized for muscle contraction and energy metabolism. In some cases, a muscle cell may contain myofibrils and myoglobins. In some cases, a muscle cell may contain a high amount of glycogen. In some cases, a muscle cell may also comprise a myocyte. In some cases, a muscle cell may develop from a myoblast. In some cases, a muscle cell may be a cardiac muscle cell, a smooth muscle cell, or a skeletal muscle cell. In some instances, a muscle cell may comprise a myofiber, a myotube, a myocyte, a myoblast, a spheroid, or a muscle cell progenitor.

In some instances, a cell may comprise an animal cell. An animal cell may comprise a cell isolated or derived from an organism from the kingdom Animalia. An animal cell may be isolated from an animal. A cell may also be an animal cell if the closest counterpart of its genome is from an animal or an animal cell.

In some instances, an animal cell may comprise a mammalian cell, a bird cell, or a fish cell, a mollusk cell, or an amphibian cell. In some instances, an animal cell may comprise a mollusk cell. In some instances, an animal cell may comprise an amphibian cell. In some instances, an animal cell may comprise a mollusk cell.

In some instances, an animal cell may comprise a mammalian cell. In some instances, an animal cell may comprise a bird cell. In some instances, an animal cell may comprise a fish cell. In some instances, an animal cell may comprise a reptilian cell. In some instances, an animal cell may comprise an invertebrate cell. In some instances, an animal cell may comprise an invertebrate cell. In some instances, an animal cell may comprise a mollusk cell.

In some instances, a mammalian cell may comprise a porcine cell, a bovine cell, a bubaline cell, an ovine cell, a caprine cell, a cervine cell, a bisontine cell, a cameline cell, an elaphine cell, or a lapine cell. In some cases, a cell may comprise a porcine cell. In some cases, a cell may comprise a bovine cell. In some cases, a cell may comprise a bubaline cell. In some cases, a cell may comprise an ovine cell. In some cases, a cell may comprise a caprine cell. In some cases, a cell may comprise a cervine cell. In some cases, a cell may comprise a bisontine cell. In some cases, a cell may comprise a cameline cell. In some cases, a cell may comprise an elaphine cell. In some cases, a cell may comprise a lapine cell.

In some instances, a bird cell may comprise an anatine cell, a galline cell, an anserine cell, a meleagrine cell, a struthionine cell, or a phasianine cell. In some cases, a cell may comprise an anatine cell. In some cases, a cell may comprise a galline cell. In some cases, a cell may comprise an anserine cell. In some cases, a cell may comprise a meleagrine cell. In some cases, a cell may comprise a struthionine cell. In some cases, a cell may comprise a phasianine cell.

In some instances, a differentiated cell may undergo self-renewal. In other cases, a differentiated cell may not undergo self-renewal. In some cases, a terminally differentiated cell may not undergo self-renewal. In some cases, a differentiated cell may not be at a pluripotent cell state.

In some cases, a cell may comprise a stem cell. In other cases, a stem cell may be at a totipotent cell state. In some cases, a stem cell maybe at a pluripotent cell state. In other cases, a stem cell may be at a multipotent cell state. In other cases, a stem cell may be at an omnipotent cell state. In other cases, a stem cell may be at a unipotent cell state. In some cases, a stem cell may comprise an induced pluripotent stem cell (iPSC). In some cases, a stem cell may comprise an embryonic stem cell, an immortalized stem cell, a mesenchymal stem cell, and/or a muscle progenitor cell. In some cases, a stem cell may comprise an embryonic stem cell, an immortalized stem cell, a mesenchymal stem cell, an iPSC, and/or a muscle progenitor cell. In some cases, a stem cell may comprise an embryonic stem cell. In some cases, a stem cell may comprise an immortalized stem cell. In some cases, a stem cell may comprise a mesenchymal stem cell. In some cases, a stem cell may comprise a muscle progenitor cell. A stem cell may comprise a reprogrammed cell. Cellular reprogramming may be a process that reverses the developmental potential of a cell or population of cells (e.g., a somatic cell). Reprogramming may be a process of driving a cell to a state with higher developmental potential, such as driving a cell backwards to a less differentiated state. The cell to be reprogrammed can be either partially or terminally differentiated prior to reprogramming. Reprogramming may infer a complete or partial reversion of the differentiation state, such as an increase in the developmental potential of a cell, to that of a cell having a pluripotent state, driving a somatic cell to a pluripotent state, such that the cell has the developmental potential of an embryonic stem cell, such as an embryonic stem cell phenotype, or may encompass a partial reversion of the differentiation state or a partial increase of the developmental potential of a cell, such as a somatic cell or a unipotent cell, to a multipotent state. Reprogramming may also encompass a partial reversion of the differentiation state of a cell to a state that renders the cell more susceptible to complete reprogramming to a pluripotent state when subjected to additional manipulations.

In some cases, iPSCs may comprise any cells obtained by re-programming of adult somatic cells which are endowed with pluripotency, a cell being capable of differentiating into the three embryonic germ cell layers, the endoderm, ectoderm and mesoderm. Such adult cells may be obtained from any adult somatic tissue (e.g., a skin fibroblast or blood cells) and undergo reprogramming by integrative genetic manipulation or non-integrative protein expression methods, which reset the cell to acquire stem cell-like characteristics. iPSCs may be formed through such processes that reverses the development of the cell or population of cells (e.g., a somatic cell) thus resulting in a naive cell type. An iPSC may be a cell that has undergone a process of driving a cell to a naive state with higher developmental and proliferation potential, such as a cell that is reset into a less differentiated state. The somatic cell, prior to induction to an iPSC, can be either partially or terminally differentiated. There may be a complete or partial reversion of the differentiation state, i.e., an increase in the developmental potential of a cell, to that of a cell having a pluripotent state. A somatic cell may be driven to a pluripotent state, such that the cell has the developmental potential of an embryonic stem cell, similar to an embryonic stem cell phenotype. Induction of a somatic cell may also encompass a partial reversion of the differentiation state or a partial increase of the developmental potential of a cell, such as a somatic cell or a unipotent cell, to a multipotent state. Induction may also encompass partial reversion of the differentiation state of a cell to a state that renders the cell more susceptible to complete induction to a pluripotent state when subjected to additional manipulations.

### Various Steps of the Methods

In some instances, the method may comprise (a) contacting a cell with an engineered nucleic acid; (b) expressing a polypeptide encoded by the engineered nucleic acid by the cell; (c) growing the cell; (d) harvesting the cell, the polypeptide encoded by the engineered nucleic acid, a molecule synthesized by the cell, or any combination thereof. In some instances, the method may comprise (a) contacting a cell with an engineered nucleic acid; (b) expressing a polypeptide encoded by the engineered nucleic acid by the cell; (c) growing the cell; (d) generating a conditioned medium comprising the cell, a polypeptide derived thereof, a metabolite derived thereof, or any combination thereof. The method may comprise (a) prior to (b), (c), (d), or a combination thereof. The method may comprise (b) subsequent to (a). The method may comprise (b) prior to (c), (d), or a combination thereof. The method may comprise (c) prior to (a), (b), or a combination thereof. The method may comprise (c) prior to (d).

In other some, the method may comprise (a) contacting a cell with an engineered nucleic acid; (b) expressing a polypeptide encoded by the engineered nucleic acid by the cell; (c) growing the cell; (d) converting the cell into a meat product. The method may comprise (a) prior to (b), (c), (d), or a combination thereof. The method may comprise (b) subsequent to (a). The method may comprise (b) prior to (c), (d), or a combination thereof. The method may comprise (c) prior to (a), (b), or a combination thereof. The method may comprise (c) prior to (d).

In some cases, the method may comprise (a) contacting a cell with an engineered nucleic acid; (b) expressing a polypeptide encoded by the engineered nucleic acid by the cell; (c) growing the cell; (d) generating a conditioned medium from the cell; (e) growing a second cell using the conditioned medium; (f) converting the second cell into a meat product. The method may comprise (a) prior to (b), (c), (d), (e), (f), or a combination thereof. The method may comprise (b) subsequent to (a). The method may comprise (b) prior to (c), (d), (e), (f) or a combination thereof. The method may comprise (c) prior to (a), (b), or a combination thereof. The method may comprise (c) prior to (d), (e), an (f).

### EXAMPLES

These examples are provided for illustrative purposes only and not to limit the scope of the claims provided herein.

### Example 1. Introduction of Engineered Nucleic Acids into Cell Populations

Provided herein are methods for introducing engineered nucleic acids into cell populations.

Precoat tissue culture plates are treated with a matrix material (e.g., geltrex, Matrigel, or vitronectin). About 15,000 iPSCs are seeded per well of a 24 well plate (adjust accordingly). The plates are incubated at 37° C., 5% CO2, for 24 hours before conducting transfection.

CleanCap® EGFP mRNA (TriLink Biotechnologies, Product #L-7601, 1 μg/μl) is used as transfection control. For introducing EGFP mRNA vector into a cell, the dosage is about 0.5 μg per well. For transfecting saRNA vector into a cell, the dosage range is from about 1 μg to 10 μg per well.

Polyplex is prepared for transfection as follows: saRNA/mRNA vectors are diluted in sterile PBS (pH 7.2), Sterile saccharide solution is added to the diluted saRNA/mRNA vectors. Once mixed thoroughly, cationic lipid or surfactant is added to the mixture. The mixture is stirred until the formation of the transfection polyplex. The polyplex is diluted with nutrient media. The diluted polyplex solution is added to the cells and pre-incubated for 20-60 minutes. The nutrient media are further diluted and incubated at 37° C. and 5% atmospheric CO2.

### Example 2. Vectors Comprising Engineered Nucleic Acids

Provided herein are vectors comprising engineered nucleic acids.

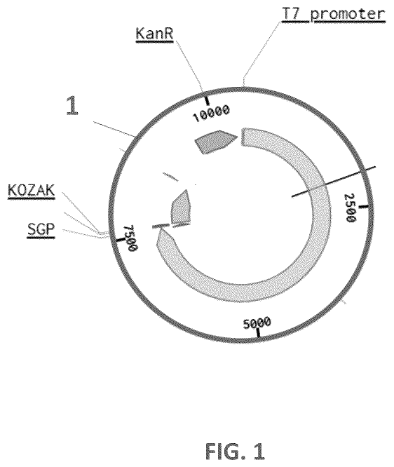

A vector harboring an engineered nucleic acid may comprise an exemplary vector of FIG. 1. The vector may comprise a T7 promoter for in vitro transcription of the Venezuelen Equine Encephalitis (VEE) nonstructural protein 1-4 (nsp1-4) coding sequences (used, for example, for the self-amplification of an saRNA). A coding sequence (“1”) comprising a polypeptide described elsewhere in this disclosure is inserted 3′ of the subgenomic promoter (SGP) and the Kozak sequence for induction of transcription and translation of the coding sequence, respectively. The vector is transfected into a cell using methods described in Example 1. The kanamycin resistance coding sequence (KanR) allows for selection of host cells (e.g., bacteria) that have taken up the vector for replication, using kanamycin selection. The plasmid is then isolated and used as a template for in vitro transcription.

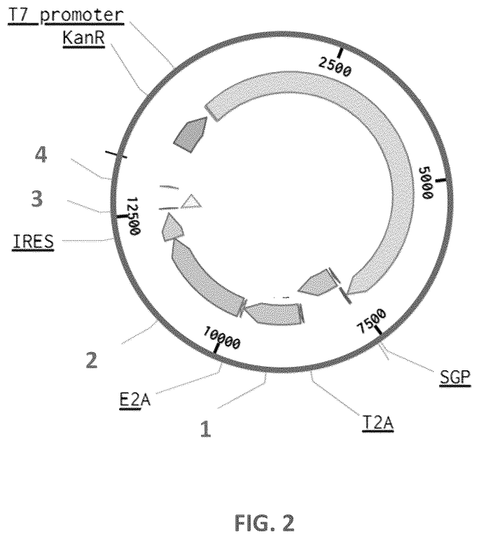

A vector harboring an engineered nucleic acid may comprise another exemplary vector of FIG. 2. The vector may comprise a T7 promoter for in vitro transcription of VEE nsp1-4 coding sequences (used, for example, for the self-amplification of an saRNA). Multiple coding sequences (“1”-“4”) comprising different polypeptides or different copies of polypeptides described elsewhere in this disclosure are inserted 3′ of the subgenomic promoter (SGP) and the Kozak sequence (not shown) for induction of transcription and translation of the coding sequence(s), respectively. In some cases, the translation of the coding sequence can be initiated via cap-dependent or cap-independent manners. For cap-independent translation, translation can be initiated using Internal Ribosome Entry Site (IRES). Coding sequence encoding 2A self-cleavage peptide, such as T2A or E2A shown in FIG. 2, or others described elsewhere in this disclosure, can be inserted between two coding sequences, so that during translation, the two coding sequences are translated into two independent peptides. The presence/absence, identity, and locations of the sequences encoding the 2A self-cleavage peptides can vary such that they are constructed differently than those shown in FIG. 2, depending on the applications of the transfected cells or the identities of the coding sequences. The vector is transfected into a cell using methods described in Example 1. The kanamycin resistance coding sequence (KanR) allows for selection of host cells (e.g., bacteria) that have taken up the vector for replication, using kanamycin selection. The plasmid is then isolated and used as a template for in vitro transcription

### Example 3. Generating Polypeptides by Using Cells Comprising Engineered RNAs

Provided herein are methods and compositions described herein for generating polypeptides encoded by engineered nucleic acids using cells.

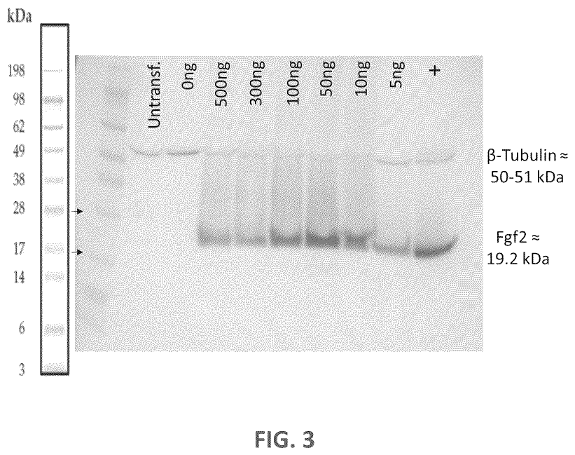

FIG. 3 depicts that saRNA could be used to produce detectable FGF2 protein across a wide titration range. 5000 porcine iPSC cells per well were seeded into a Vitronectin (VTN) coated 24 well plate. 48 hours later, the cells were transfected with varying amounts of porcine-FGF2 expressing saRNA (between 5-500 ng; labeled as 5 ng, 10 ng, 50 ng, 100 ng, 300 ng, and 500 ng in FIG. 3), or with transfection reagent alone without any saRNA (JetMessenger reagent (Polyplus); labeled as 0 ng in FIG. 3) or left without transfection (labeled as Untransf. In FIG. 3). 24 hours later, cells were harvested in RIPA buffer. 40 μg of protein for each transfection reaction was loaded per lane for western blotting. HEK cells transfected with episomal porcine FGF2 were loaded as a positive control (labeled as “+” in FIG. 3). Blue Plus2 prestained ladder (Invitrogen) was loaded into the first lane from the left. FGF2 protein was probed with rabbit anti-FGF2 antibody (ab208687, Abcam) and goat anti-rabbit HRP secondary. A clear band was detected in all lanes transfected with FGF2-saRNA and the HEK+control sample close to the target size of 19 kilo Dalton (kDa), indicating that detectable FGF2 protein was produced across this range of saRNA titration. Subsequently β-Tubulin was probed as a loading control.

The result shows that the methods and compositions described herein using cells comprising engineered RNA can generate a significant amount of polypeptides encoded by the RNA.

### Example 4. Secreting Polypeptides by Using Cells Comprising Engineered RNAs

Provided herein are methods and compositions described herein for secreting polypeptides encoded by engineered nucleic acids into culture media using cells.

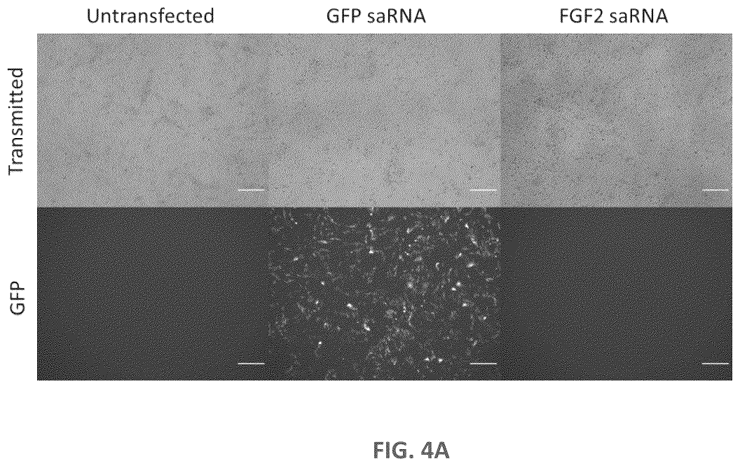

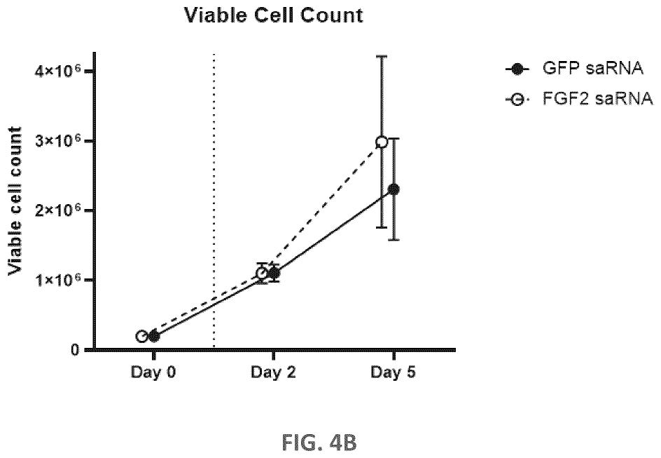

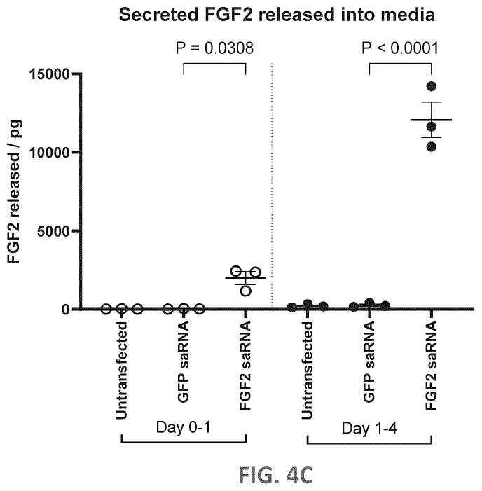

FIG. 4A-4C depict that cells transfected with saRNA encoding porcine FGF2 could lead to proliferation of the transfected cell and the secretion of FGF2 proteins into the culture media. 200 000 porcine iPSC/well were seeded into VTN coated 3×6 well plates in 2 mL growth media lacking the addition of FGF2. 24 hours later, cells were transfected with 1 μg of GFP-saRNA, porcine-FGF2 expressing saRNA or left untransfected (n=3). 4 hours post transfection, culture media were changed to remove the excess transfection reagent (JetMessenger, Polyplus)—this time point was considered transfection day 0. The next day GFP fluorescence was imaged in all wells—and confirmed in the GFP transfected cells as shown in FIG. 4A (scale-bar=300 μm). The culture media was stored for ELISA, and the cells which were now confluent were passaged into 3×new 6 well plates at 200 000 cells/well. 3 days later media was again stored for ELISA. Cells transfected with the saRNA grew an average of 11.5× with GFP saRNA and 15× with FGF2 saRNA (mean cell count at each time point shown with standard error), as shown in FIG. 4B. The dashed line at day 1 represents the point of transfection with the saRNAs. Sandwich ELISA was conducted with the bovine FGF2 kit (EB2RB, Invitrogen). This method was previously confirmed to detect the porcine FGF2 with episomally transfected HEK cells versus untransfected HEKs (data not shown; while this assay was expected to quantify the relative amount of free FGF2 available in the harvested media it would also likely be an underestimate as it would not measure the FGF2 bound to the cultured cell's receptors). As shown in FIG. 4C, the amount of FGF2 harvested from the culture media at day 4 was normalized to account for the ratio of cells which were passaged. Cells which were transfected with the FGF2-saRNA secreted significantly more FGF2 into the culture medium than that of the cells transfected with the GFP-saRNA control at day 1 (p.adj=0.308, one-way ANOVA with Šídák's multiple comparisons test) and day 4 (p.adj <0.0001, one-way ANOVA with Šídák's multiple comparisons test).

The result shows that the methods and compositions described herein using cells comprising engineered RNA can generate a significant amount of polypeptides encoded by the RNA.

### Example 5. Culturing Cells with Conditioned Media Generated from Cells Comprising Engineered Nucleic Acids

Provided herein are methods and compositions described herein for culturing cells with conditioned media generated from cells comprising engineered nucleic acids.

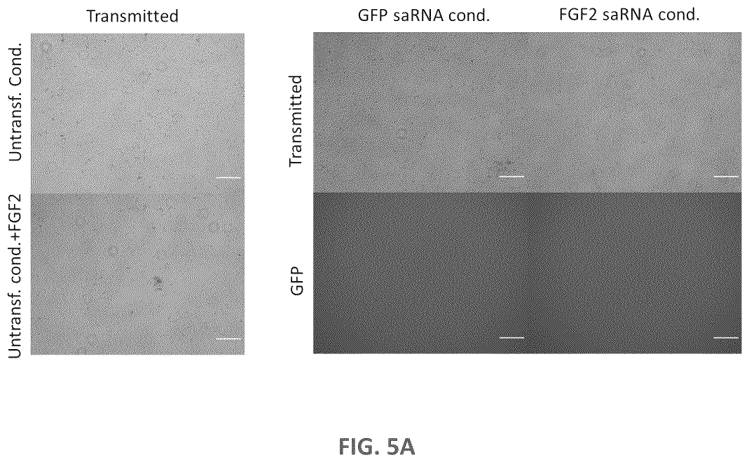

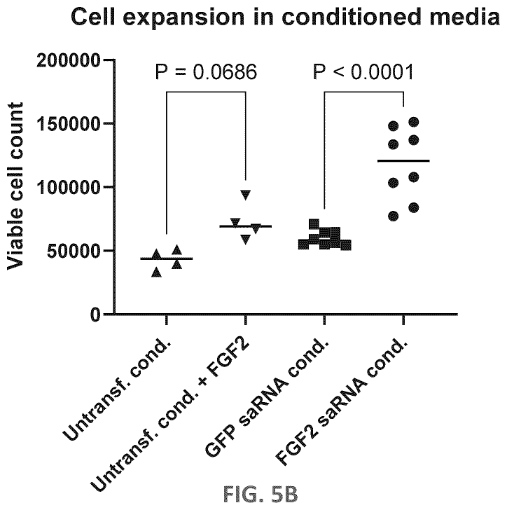

FIGS. 5A-5B depict that conditioned media generated from Example 4 was able to allow cells to proliferate without growth factors. The conditioned media were diluted 1:2 with fresh growth media without additional FGF2, and passed through a 0.22 μm filter. 6000 fresh porcine iPSC were plated in growth media without FGF2 into 24 well plates coated with VTN. Cells were left to settle for 1.5 hours before the conditioned media was removed and replaced with the conditioned media from the untransfected wells (4 wells; labeled as Untransf. Cond. In FIG. 5B), untransfected wells plus 40 ng/ml recombinant bovine/porcine FGF2 (Qkine) (4 wells; labeled as Untransf. Cond.+FGF2 In FIG. 5B), GFP-saRNA transfected wells (8 wells; labeled as GFP saRNA cond. In FIG. 5B) or FGF2-saRNA transfected wells; labeled as FGF2 saRNA cond. In FIG. 5B. Cells grew for the next 2 days and were imaged to record cell density and confirm that no GFP expression had carried over indicating saRNA-transfection complexes were unlikely to have been transferred, as shown in FIG. 5A (scale-bar=300 μm). Cells were harvested with TrypLE Express (Gibco), and counted in triplicate using the Chemotec Nucleocounter NC-250 cell viability assay and protocol with solution 18, and averaged per well/24 well plate. FIG. 5B shows that porcine iPSCs grown with the FGF2-saRNA transfected cell conditioned media to almost twice the density in this time as the GFP-saRNA conditioned media control, (p.adj <0.0001, one-way ANOVA with Šídák's multiple comparisons test).

The result shows that the methods and compositions described herein using cells comprising engineered RNA can generate media that can support cell growth in the medium without needing other exogenously synthesized or purified growth factors.

### Example 6. Growing or Culturing a Cell with an Engineered RNA

Provided herein are methods and compositions described herein for growing or culturing cells that have engineered nucleic acids.

About 200 000 cells/well are seeded into VTN coated 3×6 well plates in 2 mL growth media lacking the addition of a growth factor. 24 hours later, cells are transfected with 1 μg of engineered nucleic acid encoding the growth factor, control, or left untransfected (n=3). 4 hours post transfection, culture media are changed to remove the excess transfection reagent (JetMessenger, Polyplus)—this time point is considered transfection day 0. The number of cells are then counted in the following days to determine the proliferation of the transfected cells and controls.

While preferred embodiments of the present disclosure have been shown and described herein, it may be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions may now occur to those skilled in the art without departing from the disclosure. Furthermore, it shall be understood that all aspects of the disclosure are not limited to the specific depictions, configurations or relative proportions set forth herein which depend upon a variety of conditions and variables. It may be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1.-159. (canceled)

160. A method, comprising:
(a) contacting a cell with an engineered ribonucleic acid (RNA); and
(b) growing said cell within a culture medium,
wherein said culture medium comprises at most about 100 micrograms per milliliter (μg/mL) exogenous growth factor, wherein said engineered RNA encodes a polypeptide that allows said cell to be cultured within said culture medium, and wherein said engineered RNA or a derivative thereof is not integrated into a genome of said cell.

161. The method of claim 160, wherein said engineered RNA comprises a messenger ribonucleic acid (mRNA), a self-amplifying RNA (saRNA), an endless/circular RNA (eRNA), a trans-amplifying RNA (ta-RNA), or any combinations thereof.

162. The method of claim 161, wherein said engineered RNA comprises said saRNA.

163. The method of claim 160, further comprising expressing said polypeptide encoded by said engineered RNA or said derivative thereof by said cell.

164. The method of claim 163, further comprising harvesting said polypeptide from said culture medium.

165. The method of claim 163, further comprising generating a conditioned medium comprising said polypeptide.

166. The method of claim 165, further comprising growing a second cell different from said cell using said conditioned medium.

167. The method of claim 163, wherein said cell secretes said polypeptide.

168. The method of claim 167, wherein said polypeptide is encapsulated by an endogenous vesicle derived from said cell.

169. The method of claim 160, wherein said growing in (b) generates a population of said cell.

170. The method of claim 169, wherein a progeny of said cell within said population of said cell is cultured within said culture medium by using a second polypeptide derived from said cell.

171. The method of claim 160, wherein said polypeptide comprises at least a sequence comprising: (1) at least 70% sequence identity to said exogenous growth factor, (2) at least 70% sequence identity to a growth factor receptor activated by said exogenous growth factor, or (3) a combination of (1)-(2).

172. The method of claim 160, wherein said engineered RNA encodes a polypeptide comprising a growth factor, a growth factor receptor, a cell proliferation signaling pathway regulator, a cell-cycle regulator, an immune response regulator, a hormone, a Maillard reaction peptide, a metallopeptide, or any combinations thereof.

173. The method of claim 160, wherein said engineered RNA encodes Basic Fibroblast Growth Factor (FGF), FGF-2, FGF-7, Soluble IFN Alpha/Beta Receptor B18 (B18R), Transforming Growth Factor Beta (TGF-beta), TGF-beta 1, Neuregulin 1 (NRG1), Heregulin, Insulin, Insulin-like Growth Factor (IGF), IGF-1, Transferrin, Epidermal Growth Factor (EGF), Activin, Nodal, Wnt, Albumin, myoglobin, Haemoglobin, Leukaemia Inhibitory Factor, Bone Morphogenic Protein (BMP), BMP-2, BMP-7, Platelet-Derived Growth Factor (PDGF), PDGF-BB, Vascular Endothelial Growth Factor (VEGF), VEGF-A, Hepatocyte Growth Factor (HGF), Bovine Serum Albumin, Immune Evasion Peptide E3, Immune Evasion Peptide K3, any functional variants thereof, or any combinations thereof.

174. The method of claim 160, wherein said cell comprises at least two engineered RNAs.

175. The method of claim 160, wherein said engineered RNA comprises an alphavirus replication sequence, a Kozac sequence, a poly-A tail, a 5′cap, an internal Ribosome Entry Sequence (IRES), a sequence encoding a 2A self-cleaving peptide, a 26S subgenomic promoter, or any combinations thereof.

176. The method of claim 160, wherein said engineered RNA comprises at least one copy of a coding sequence.

177. The method of claim 160, wherein said engineered RNA comprises at most one engineered RNA.

178. The method of claim 160, wherein said culture medium does not comprise any exogenous growth factors.

179. The method of claim 160, wherein said culture medium comprise at least 1 exogenous growth factor.
